SEQUENCE LISTING (36 sequences)

SEQ ID NO: 1 - protein, 591 aa, Homo sapiens
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - DNA, 2032 bp, Homo sapiens
tccatattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatg

SEQ ID NO: 3 - protein, 8 aa, Homo sapiens
EMTPVNPG

SEQ ID NO: 4 - protein, 34 aa, Homo sapiens
LSEDKLLACGEGAADIIIGHLCIRHEMTPVNPGV

SEQ ID NO: 5 - protein, 197 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRES

SEQ ID NO: 6 - protein, 192 aa, Homo sapiens
SLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 7 - protein, 201 aa, Homo sapiens
QALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 8 - protein, 389 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 9 - protein, 393 aa, Homo sapiens
SLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 10 - protein, 331 aa, Homo sapiens
QDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 11 - protein, 1032 aa, Homo sapiens [sequence omitted]

SEQ ID NO: 12 - DNA, 6082 bp, Homo sapiens
ataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccggttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaattacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacacgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaa

SEQ ID NO: 13 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 14 - DNA, 2757 bp, Homo sapiens
gacaagtactgagtgaactcaaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtcccacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggtgttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataa

SEQ ID NO: 15 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMICLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCNNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 16 - DNA, 3199 bp, Homo sapiens
gtttttctaaacagcctgacactgaggggaggcagtgagactgtaagcagtctgggttgggcagaaggcagaaaaccagcagagtcacagaggagatggccaactgccaaatagccatcttgtaccagagattccagagagtggtctttggaatttcccaactcctttgcttcagtgccctgatctctgaactaacaaaccagaaagaagtggcagcatggacttatcattacagcacaaaagcatactcatggaatatttcccgtaaatactgccagaatcgctacacagacttagtggccatccagaataaaaatgaaattgattacctcaataaggtcctaccctactacagctcctactactggattgggatccgaaagaacaataagacatggacatgggtgggaaccaaaaaggctctcaccaacgaggctgagaactgggctgataatgaacctaacaacaaaaggaacaacgaggactgcgtggagatatacatcaagagtccgtcagcccctggcaagtggaatgatgagcactgcttgaagaaaaagcacgcattgtgttacacagcctcctgccaggacatgtcctgcagcaaacaaggagagtgcctcgagaccatcgggaactacacctgctcctgttaccctggattctatgggccagaatgtgaatacgtgagagagtgtggagaacttgagctccctcaacacgtgctcatgaactgcagccaccctctgggaaacttctcttttaactcgcagtgcagcttccactgcactgacgggtaccaagtaaatgggcccagcaagctggaatgcttggcttctggaatctggacaaataagcctccacagtgtttagctgcccagtgcccacccctgaagattcctgaacgaggaaacatgatctgccttcattctgcaaaagcattccagcatcagtctagctgcagcttcagttgtgaagagggatttgcattagttggaccggaagtggtgcaatgcacagcctcgggggtatggacagccccagccccagtgtgtaaagctgtgcagtgtcagcacctggaagcccccagtgaaggaaccatggactgtgttcatccgctcactgcttttgcctatggctccagctgcaaatttgagtgccagcccggctacagagtgaggggcttggacatgctccgctgcattgactctggacactggtctgcacccttgccaacctgtgaggctatttcgtgtgagccgctggagagtcctgtccacggaagcatggattgctctccatccttgagagcgtttcagtatgacaccaactgtagcttccgctgtgctgaaggtttcatgctgagaggagccgatatagttcggtgtgataacttgggacagtggacagcaccagccccagtctgtcaagctttgcagtgccaggatctcccagttccaaatgaggcccgggtgaactgctcccaccccttcggtgcctttaggtaccagtcagtctgcagcttcacctgcaatgaaggcttgctcctggtgggagcaagtgtgctacagtgcttggctactggaaactggaattctgttcctccagaatgccaagccattccctgcacacctttgctaagccctcagaatggaacaatgacctgtgttcaacctcttggaagttccagttataaatccacatgtcaattcatctgtgacgagggatattctttgtctggaccagaaagattggattgtactcgatcgggacgctggacagactccccaccaatgtgtgaagccatcaagtgcccagaactctttgccccagagcagggcagcctggattgttctgacactcgtggagaattcaatgttggctccacctgtcatttctcttgtaacaatggctttaagctggaggggcccaataatgtggaatgcacaacttctggaagatggtcagctactccaccaacctgcaaaggcatagcatcacttcctactccagggttgcaatgtccagccctcaccactcctgggcagggaaccatgtactgtaggcatcatccgggaacctttggttttaataccacttgttactttggctgcaacgctggattcacactcataggagacagcactctcagctgcagaccttcaggacaatggacagcagtaactccagcatgcagagctgtgaaatgctcagaactacatgttaataagccaatagcgatgaactgctccaacctctggggaaacttcagttatggatcaatctgctctttccattgtctagagggccagttacttaatggctctgcacaaacagcatgccaagagaatggccactggtcaactaccgtgccaacctgccaagcaggaccattgactatccaggaagccctgacttactttggtggagcggtggcttctacaataggtctgataatgggtgggacgctcctggctttgctaagaaagcgtttcagacaaaaagatgatgggaaatgccccttgaatcctcacagccacctaggaacatatggagtttttacaaacgctgcatttgacccgagtccttaaggtttccataaacacccatgaatcaaagacatggaattaccttagattagctctggaccagcctgttggacccgctctggaccaaccctgtttcctgagtttgggattgtggtacaatctcaaattctcaacctaccaccccttcctgtcccacctcttctcttcctgtaacacaagccacagaagccaggagcaaatgtttctgcagtagtctctgtgctttgactcacctgttacttgaaataccagtgaaccaaagagactggagcatctgactcacaagaagaccagactgtggagaaataaaaatacctctttattttttgattgaaggaaggttttctccactttgttggaaagcaggtggcatctctaattggaagaaattcctgtagcatcttctggagtctccagtggttgctgttgatgaggcctcttggacctctgctctgaggcttccagagagtcctctggatggcaccagaggctgcagaaggccaagaatcaagctagaaggccacatgtcaccgtggaccttcctgccaccagtcactgtccctcaaatgacccaaagaccaatattcaaatgcgtaattaaaagaattttcccc

SEQ ID NO: 17 - protein, 381 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSENVALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 18 - DNA, 2811 bp, Homo sapiens
ggggagtcgggggcagcagcaagatgcgaagcgagccgtacagatcccgggctctccgaacgcaacttcgccctgcttgagcgaggctgcggtttccgaggccctctccagccaaggaaaagctacacaaaaagcctggatcactcatcgaaccacccctgaagccagtgaaggctctctcgcctcgccctctagcgttcgtctggagtagcgccaccccggcttcctggggacacaggtttggcaccatggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaagtcgctggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcctagaactggaagctgtccacccaccggaagcgctctttacttggtcgctggccaccccagtgtttggaaaaaaatctctgggcttcgactgctgccagggaggagctgctgcaagccagagggaggaagggggagaatacgaacagcctggtggtgtcgggtgttggtgggtagagttagttcctgtgaacaatgcactgggaagggtggagatcaggtcccggcctggaatatattttctacccccctggagctttgattttgcactgagccaaaggtctagcattgtcaagctcctaaagggttcatttggcccctcctcaaagactaatgtccccatgtgaaagcgtctctttgtctggagctttgaggagatgttttccttcactttagtttcaaacccaagtgagtgtgtgcacttctgcttctttagggatgccctgtacatcccacaccccaccctcccttcccttcatacccctcctcaacgttcttttactttatactttaactacctgagagttatcagagctggggttgtggaatgatcgatcatctatagcaaataggctatgttgagtacgtaggctgtgggaagatgaagatggtttggaggtgtaaaacaatgtccttcgctgaggccaaagtttccatgtaagcgggatccgttttttggaatttggttgaagtcactttgatttctttaaaaaacatcttttcaatgaaatgtgttaccatttcatatccattgaagccgaaatctgcataaggaagcccactttatctaaatgatattagccaggatccttggtgtcctaggagaaacagacaagcaaaacaaagtgaaaaccgaatggattaacttttgcaaaccaagggagatttcttagcaaatgagtctaacaaatatgacatctgtctttggcacttttgttgatgtttatttcagaatgttgtgtgattcatttcaagcaacaacatggttgtattttgttgtgttaaaagtacttttcttgatttttgaatgtatttgtttcagcagaagtcattttattggatttttctaacccgtgttaacaccattgaatgtgtatttcttaagaaaataccaccctcttgtgcccttaaaagcattactttaactggtagggaacgccagaaacttttcagtccagctattcattagatagtaattgaagatatgtataaatattacaaagaataaaaatatattactgtctctttagtatggttttcagtgcaattaaaccgagagatgtcttgtttttttaaaaagaatagtatttaataggtttctgacttttgtggatcattttgcacatagctttatcaacttttaaacattaataaactgatttttttaaggaaaaaaaaaaaaaaaaaaaaaaaaaaaaaa

SEQ ID NO: 19 - protein, 742 aa, Homo sapiens [sequence omitted]

SEQ ID NO: 20 - DNA, 3091 bp, Homo sapiens
gggagacccaagcttctagagatccctcgacctcgagatccattgtgctctaaagagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcatgtggtgattccccggatccaccccaactccatctgtgcagcaaacaacacaggggtgtacatcctcacatacaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaagcctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcctttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccagagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtacaacgcttcagcctactgcaaatccaaacacaggtttggtggaaaatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttgtaaattagggcccaattaataatcagcaagaatttgatcgttcagttccacttggaggccttcatcctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttggggtgtactagttacacatcttcaacagaccccctctagaaatttttcagatgcttctgggagacaccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaaaa

SEQ ID NO: 21 - protein, 1170 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 22 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVQLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 23 - DNA, 5133 bp, Homo sapiens
cctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttggcctgtgaccctgggctgtctcgaacgtgtgaccagaacacctatctgagtggcctgtgttacctcttccgccagaatctgcagggtcccatgctgcaggggcgccctggttttcaggaatgtatcaagggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaatggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctctacccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgcagggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgaatgtctcctttgcaggctcatagggaagacctgctgagggaccagccaagagggctgcaaaagtgagggcttgtcattaccagacggttcaccagcctctcttggttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgcccacccctccttccttgaccagcagatcccagctcacgtcacacacttggttgggtcctcacatctttcacacttccaccaccctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgacgccgcatgatatgtcagggcgtgggacatctagtaggtgcttgacataatttcactgaattaatgacagagccagtgggaagatacagaaaaagagggccggggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccggacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaa

SEQ ID NO: 24 - DNA, 2291 bp, Homo sapiens
ctcgccctggtggggctgctctccctcgggtgcgtcctctctcaggagtgcacgaagttcaaggtcagcagctgccgggaatgcatcgagtcggggcccggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgcccgcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacggaaagctgggcgccatcctgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccatctcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggtccttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagca

SEQ ID NO: 25 - protein, 1170 aa, Homo sapiens; identical to SEQ ID NO: 21

SEQ ID NO: 26 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 27 - protein, 1163 aa, Homo sapiens
MTRTRAALLLFTALATSLGFNLDTEELTAFRVDSAGFGDSVVQYANSWVVVGAPQKITAANQTGGLYQCGYSTGACEPIGLQVPPEAVNMSLGLSLASTTSPSQLLACGPTVHHECGRNMYLTGLCFLLGPTQLTQRLPVSRQECPRQEQDIVFLIDGSGSISSRNFATMMNFVRAVISQFQRPSTQFSLMQFSNKFQTHFTFEEFRRTSNPLSLLASVHQLQGFTYTATAIQNVVHRLFHASYGARRDATKILIVITDGKKEGDSLDYKDVIPMADAAGIIRYAIGVGLAFQNRNSWKELNDIASKPSQEHIFKVEDFDALKDIQNQLKEKIFAIEGTETTSSSSFELEMAQEGFSAVFTPDGPVLGAVGSFTWSGGAFLYPPNMSPTFINMSQENVDMRDSYLGYSTELALWKGVQSLVLGAPRYQHTGKAVIFTQVSRQWRMKAEVTGTQIGSYFGASLCSVDVDTDGSTDLVLIGAPHYYEQTRGGQVSVCPLPRGWRRWWCDAVLYGEQGHPWGRFGAALTVLGDVNGDKLTDVVIGAPGEEENRGAVYLFHGVLGPSISPSHSQRIAGSQLSSRLQYFGQALSGGQDLTQDGLVDLAVGARGQVLLLRTRPVLWVGVSMQFIPAEIPRSAFECREQVVSEQTLVQSNICLYIDKRSKNLLGSRDLQSSVTLDLALDPGRLSPRATFQETKNRSLSRVRVLGLKAHCENFNLLLPSCVEDSVTPITLRLNFTLVGKPLLAFRNLRPMLAADAQRYFTASLPFEKNCGADHICQDNLGISFSFPGLKSLLVGSNLELNAEVMVWNDGEDSYGTTITFSHPAGLSYRYVAEGQKQGQLRSLHLTCDSAPVGSQGTWSTSCRINHLIFRGGAQITFLATFDVSPKAVLGDRLLLTANVSSENNTPRTSKTTFQLELPVKYAVYTVVSSHEQFTKYLNFSESEEKESHVAMHRYQVNNLGQRDLPVSINFWVPVELNQEAVWMDVEVSHPQNPSLRCSSEKIAPPASDFLAHIQKNPVLDCSIAGCLRFRCDVPSFSVQEELDFTLKGNLSFGWVRQILQKKVSVVSVAEITFDTSVYSQLPGQEAFMRAQTTTVLEKYKVHNPTPLIVGSSIGGLLLLALITAVLYKVGFFKRQYKEMMEEANGQIAPENGTQTPSPPSEK

SEQ ID NO: 28 - protein, 769 aa, Homo sapiens; identical to SEQ ID NO: 22

SEQ ID NO: 29 - DNA, 5133 bp, Homo sapiens; identical to SEQ ID NO: 23

SEQ ID NO: 30 - DNA, 4742 bp, Homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 31 - DNA, 4666 bp, Homo sapiens
agtaccttggtccagctcttcctgcaacggcccaggagctcagagctccacatctgaccttctagtcatgaccaggaccagggcagcactcctcctgttcacagccttagcaacttctctaggtttcaacttggacacagaggagctgacagccttccgtgtggacagcgctgggtttggagacagcgtggtccagtatgccaactcctgggtggtggttggagccccccaaaagataacagctgccaaccaaacgggtggcctctaccagtgtggctacagcactggtgcctgtgagcccatcggcctgcaggtgcccccggaggccgtgaacatgtccctgggcctgtccctggcgtctaccaccagcccttcccagctgctggcctgcggccccaccgtgcaccacgagtgcgggaggaacatgtacctcaccggactctgcttcctcctgggccccacccagctcacccagaggctcccggtgtccaggcaggagtgcccaagacaggagcaggacattgtgttcctgatcgatggctcaggcagcatctcctcccgcaactttgccacgatgatgaacttcgtgagagctgtgataagccagttccagagacccagcacccagttttccctgatgcagttctccaacaaattccaaacacacttcactttcgaggaattcaggcgcagctcaaaccccctcagcctgttggcttctgttcaccagctgcaagggtttacatacacggccaccgccatccaaaatgtcgtgcaccgattgttccatgcctcatatggggcccgtagggatgccgccaaaattctcattgtcatcactgatgggaagaaagaaggcgacagcctggattataaggatgtcatccccatggctgatgcagcaggcatcatccgctatgcaattggggttggattagcttttcaaaacagaaattcttggaaagaattaaatgacattgcatcgaagccctcccaggaacacatatttaaagtggaggactttgatgctctgaaagatattcaaaaccaactgaaggagaagatctttgccattgagggtacggagaccacaagcagtagctccttcgaattggagatggcacaggagggcttcagcgctgtgttcacacctgatggccccgttctgggggctgtggggagcttcacctggtctggaggtgccttcctgtaccccccaaatatgagccctaccttcatcaacatgtctcaggagaatgtggacatgagggactcttacctgggttactccaccgagctggccctctggaaaggggtgcagagcctggtcctgggggccccccgctaccagcacaccgggaaggctgtcatcttcacccaggtgtccaggcaatggaggatgaaggccgaagtcacggggactcagatcggctcctacttcggggcctccctctgctccgtggacgtagacagcgacggcagcaccgacctggtcctcatcggggccccccattactacgagcagacccgagggggccaggtgtctgtgtgtcccttgcccagggggtggagaaggtggtggtgtgatgctgttctctacggggagcagggccacccctggggtcgctttggggcggctctgacagtgctgggggatgtgaatggggacaagctgacagacgtggtcatcggggccccaggagaggaggagaaccggggtgctgtctacctgtttcacggagtcttgggacccagcatcagcccctcccacagccagcggatcgcgggctcccagctctcctccaggctgcagtattttgggcaggcactgagcgggggtcaagacctcacccaggatggactggtggacctggctgtgggggcccggggccaggtgctcctgctcaggaccagacctgtgctctgggtgggggtgagcatgcagttcatacctgccgagatccccaggtctgcgtttgagtgtcgggagcaggtggtctctgagcagaccctggtacagtccaacatctgcctttacattgacaaacgttctaagaacctgcttgggagccgtgacctccaaagctctgtgaccttggacctggccctcgaccctggccgcctgagtccccgtgccaccttccaggaaacaaagaaccggagtctgagccgagtccgagtcctcgggctgaaggcacactgtgaaaacttcaacctgctgctcccgagctgcgtggaggactctgtgacccccattaccttgcgtctgaacttcacgctggtgggcaagcccctccttgccttcagaaacctgcggcctatgctggccgccgatgctcagagatacttcacggcctccctaccctttgagaagaactgtggagccgaccatatctgccaggacaatctcggcatctccttcagcttcccaggcttgaagtccctgctggtggggagtaacctggagctgaacgcagaagtgatggtgtggaatgacggggaagactcctacggaaccaccatcaccttctcccaccccgcaggactgtcctaccgctacgtggcagagggccagaaacaagggcagctgcgttccctgcacctgacatgtgacagcgccccagttgggagccagggcacctggagcaccagctgcagaatcaaccacctcatcttccgtggcggcgcccagatcaccttcttggctacctttgacgtctcccccaaggctgtcctgggagaccggctgcttctgacagccaatgtgagcagtgagaacaacactcccaggaccagcaagaccaccttccagctggagctcccggtgaagtatgctgtctacactgtggttagcagccacgaacaattcaccaaatacctcaacttctcagagtctgaggagaaggaaagccatgtggccatgcacagataccaggtcaataacctgggacagagggacctgcctgtcagcatcaacttctgggtgcctgtggagctgaaccaggaggctgtgtggatggatgtggaggtctcccacccccagaacccatcccttcggtgctcctcagagaaaatcgcacccccagcatctgacttcctggcgcacattcagaagaatcccgtgctggactgctccattgctggctgcctgcggttccgctgtgacgtcccctccttcagcgtccaggaggagctggatttcaccctgaagggcaacctcagctttggctgggtccgccagatattgcagaagaaggtgtcggtcgtgagtgtggctgaaattacgttcgacacatccgtgtactcccagcttccaggacaggaggcatttatgagagctcagacgacaacggtgctggagaagtacaaggtccacaaccccacccccctcatcgtaggcagctccattgggggtctgttgctgctggcactcatcacagcggtactgtacaaagttggcttcttcaagcgtcagtacaaggaaatgatggaggaggcaaatggacaaattgccccagaaaacgggacacagacccccagcccgcccagtgagaaatgatcccctctttgccttggacttcttctcccccgcgagttttccccacttacttaccctcacctgtcaggcctgacggggaggaaccactgcaccaccgagagaggctgggatgggcctgcttcctgtctttgggagaaaacgtcttgcttgggaaggggcctttgtcttgtcaaggttccaactggaaacccttaggacagggtccctgctgtgttccccaaaggacttgacttgcaatttctacctagaaatacatggacaatacccccaggcctcagtctcccttctcccatgaggcacgaatgatctttctttcctttctttttttttttttttcttttcttttttttttttttgagacggagtctcgctctgtcacccaggctggagtgcaatggcgtgatctcggctcactgcaacctccgcctcccgggttcaagtaattctgctgtctcagcctcctgagtagctgggactacaggcacacgccacctcgcccggcccgatctttctaaaatacagttctgaatatgctgctcatccccacctgtcttcaacagctccccattaccctcaggacaatgtctgaactctccagcttcgcgtgagaagtccccttccatcccagagggtgggcttcagggcgcacagcatgagaggctctgtgcccccatcaccctcgtttccagtgaattagtgtcatgtcagcatcagctcagggcttcatcgtggggctctcagttccgatttcccaggctgaattgggagtgagatgcctgcatgctgggttctgcacagctggcctcccgcgttgggcaacattgctggctggaagggaggagcgccctctagggagggacatggccccggtgcggctgcagctcacccagccccaggggcagaagagacccaaccacttctattttttgaggctatgaatatagtacctgaaaaaatgccaagacatgattatttttttaaaaagcgtactttaaatgtttgtgttaataaattaaaacatgcacaaaaagatgcatctaccgctcttgggaaatatgtcaaaggtctaaaaataaaaaagccttctgtgaaaaaaaaaaaaaaaaa

SEQ ID NO: 32 - DNA, 2291 bp, Homo sapiens; identical to SEQ ID NO: 24

SEQ ID NO: 33 - skipped

SEQ ID NO: 34 - protein, 1038 aa, Homo sapiens
MFPTESAWLGKRGANPGPEAAVRETVMLLLCLGVPTGRPYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFTIVIISSSLLLGLIVLLLISYVMWKAGFFKRQYKSILQEENRRDSWSYINSKSNDD

SEQ ID NO: 35 - DNA, 3567 bp, Homo sapiens
cgccatcccgcgctctgcggactgggaggcccgggccaggacgcgagtctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctcggcggcagaaaccgggagtggggccgggcgagtgcgcggcatcccaggccggcccgaacgtccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgcttagtgttgaatgttccccaccgagagcgcatggcttgggaagcgaggcgcgaacccgggccccgaagccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccagttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagcccgccc

SEQ ID NO: 36 - protein, 591 aa, Homo sapiens
RTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVQFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

(19) United States
(12) Patent Application Publication
(10) Pub. No.: US 2010/0150915 A1
(43) Pub. Date: Jun. 17, 2010

(54) METHODS OF TREATING MULTIPLE SCLEROSIS BY ADMINISTRATION OF ALPHA-FETOPROTEIN IN COMBINATION WITH AN INTEGRIN ANTAGONIST

(76) Inventors: Edward J. Stewart, Winchester, MA (US); Michael Briskin, Lexington, MA (US)

Correspondence Address: CLARK & ELBING LLP, 101 FEDERAL STREET, BOSTON, MA, 02110, US

(21) Appl. No.: 12/527,816

(22) PCT Filed: Feb. 20, 2008
(86) PCT No.: PCT/US08/02231

§ 371 Date: Dec. 30, 2009

Related U.S. Application Data
(60) Provisional application No. 60/902,216, filed on Feb. 20, 2007.

(51) Int. Cl. A61K 39/395; A61K 38/14; A61P 37/00

(52) U.S. Cl. 424/133.1; 424/130.1; 514/8

(57) ABSTRACT

The present invention relates to methods for treating multiple sclerosis by administering therapeutically effective amounts of an alpha-fetoprotein polypeptide (or a biologically active fragment, derivative, or analog thereof) and an integrin antagonist (e.g., natalizumab) to a patient in need thereof. Also disclosed are compositions and kits that comprise therapeutically effective amounts of an alpha-fetoprotein polypeptide (or a biologically active fragment, derivative, or analog thereof) and an integrin antagonist (e.g., natalizumab).

### FIELD OF THE INVENTION

This invention relates to treatment methods using alpha-fetoprotein, including its biologically active fragments, analogs, and derivatives, in conjunction with administration of an integrin antagonist for the treatment of multiple sclerosis.

### BACKGROUND OF THE INVENTION

Multiple Sclerosis (MS) is a neurological disease first described in Holland by a 14th century physician, and which is characterized by irreversible degeneration of the nerves of the central nervous system (CNS). Although the underlying cause is unclear, the neurodegeneration in MS is the direct result of demyelination, or the stripping of myelin, a protein that normally lines the outer layer and insulates the nerves. As the condition progresses, patches of inflammation and scarring develop, interfering with the function of the nerves. Consequently, an MS patient gradually loses sensory and motor functions of the body. About 400,000 to 500,000 people in the U.S. suffer from MS. Usually, a patient is diagnosed with MS between 20 and 40 years of age, but MS has been diagnosed as early as age 15 and as late as age 60. MS is relentless and progressively destructive unless the patient receives medical therapy that is effective in halting or slowing the deterioration. While some individuals manage well in the short term, MS patients invariably become more significantly impaired by the disease over time.

Current therapies for MS are aimed at alleviating the symptoms of the disease and arresting its progress. Drug treatment usually entails the use of disease-modifying agents, such as the interferons (interferon-β-1a, β-1b, and α), glatiramer acetate, or corticosteroids, such as methylprednisolone and prednisone. Chemotherapeutic agents, such as mitoxantrone, methotrexate, azathioprine, and cladribine cyclophosphamide are also used to treat MS.

Thought to be an autoimmune disease, MS is also treated with various immunologic therapies. For example, cyclosporine, an immunosuppressive agent is used to treat MS. In addition, natalizumab (Tysabri®, Elan Corporation, Inc./Biogen-Idec), a selective adhesion molecule inhibitor introduced in 2005, has also been used for the treatment of MS. Natalizumab, a humanized anti-α4 integrin antibody, has been shown to block autoimmiune encephalomyelitis in a rat and a mouse model (Yednock et al., Nature 356:63, 1992; Baron et al., J. Exp. Med. 177:57, 1993). Many of the current treatments for MS either lack efficacy, or pose serious risks and side effects. For example, natalizumab can increase the risk of progressive multifocal leukoencephalopathy (PML), an opportunistic viral infection of the brain that typically leads to death or severe disability. Thus, there remains a need for new and effective therapeutic approaches for the treatment of MS. The present invention addresses this and other related needs.

### SUMMARY OF THE INVENTION

The present invention features compositions and methods for treating, preventing, or reducing one or more of the symptoms of MS (e.g., tingling, numbness, tremors, loss of balance, weakness in one or more limbs, blurred or double vision, slurred speech, swallowing problems, paralysis, lack or coordination, cognitive difficulties, fatigue, muscle spasms, dizziness, breathing problems, and seizures) or the progression of MS, in a patient by co-administering alpha-fetoprotein (AFP) and an integrin antagonist. The integrin antagonist can be, e.g., an antibody, a blocking peptide, a nucleic acid inhibitor, or a small molecule, as is described herein.

In a first aspect, the present invention features a method of treating a patient with MS by administering AFP (or biologically active fragment, derivative, or analog thereof) and an integrin antagonist, each in an amount (e.g., a therapeutically effective amount), to the patient.

In an embodiment, the integrin antagonist is an α4 integrin antagonist. The integrin antagonist can be, e.g., an antibody, such as natalizumab, a blocking peptide, a nucleic acid inhibitor, or a small molecule inhibitor.

Different administration schedules can be followed in the above method. For instance, the AFP (or biologically active fragment, derivative, or analog thereof) or the integrin antagonist can be administered one or more times (e.g., 1, 2, 3, 4, 5, or times or more) hourly, daily, weekly, biweekly, or monthly. In addition, the dosage of the AFP (or biologically active fragment, derivative, or analog thereof) per administration may be the same or different.

In other embodiments of the above method, the AFP (or biologically active fragment, derivative, or analog thereof), and the integrin antagonist are administered coextensively or separately. Many variations of administration schemes are possible, for example, both the AFP (or a biologically active fragment, derivative, or analog thereof) and the integrin antagonist may be administered to the patient during the first treatment phase. Subsequently, the administration of one (e.g., the AFP or the integrin antagonist) may be terminated or the dosage amount may be modified (e.g., increased or decreased) while administration of the other is continued (e.g., at the same dosage level or at a modified level (e.g., increased or decreased)). Alternatively, both the AFP (or biologically active fragment, derivative, or analog thereof) and the integrin antagonist may be administered initially at their maximal or minimal dosages with subsequent dosages of both being reduced or increased, respectively, during the treatment regimen. In addition, the AFP (or biologically active fragment, derivative, or analog thereof) may be administered prior to or following administration of the integrin antagonist.

In an additional embodiment of the method, the AFP (or a biologically active fragment, derivative, or analog thereof) and the integrin antagonist are administered in the same dosage form or separate dosage forms. In additional embodiments of the method, the AFP (or biologically active fragment, derivative, or analog thereof) is administered at a dosage in the range of 0.1 mg to 400 mg and/or the integrin antagonist is administered at a dosage in the range of 0.1 mg to 500 mg.

In another embodiment of the method, the AFP (or a biologically active fragment, derivative, or analog thereof) and the integrin antagonist are administered via the same route of administration or via two different routes of administration.

In an embodiment, the method further includes administering a supplemental agent, such as an antagonist of one or more of the following proteins: CD80, P-selectin, sphingosine-1-phosphate receptor-1, hyaluronate receptor, leukocyte function antigen-1 (LFA-1), CD11/CD18, CD20, CD86, ICOS ligand, CCR2, CXCR3, CCR5, CD40, CD154, CD28, IL-23, IL-17, and IL-6. The supplemental agent can be, e.g., an antibody, a blocking peptide, a nucleic acid inhibitor, or a small molecule inhibitor. The CD80, P-selectin, sphingosine-1-phosphate receptor-1, hyaluratone receptor, LFA-1, CD11/CD18, CD20, CD86, ICOS ligand, CCR2, CXCR3, CCR5, CD40, CD154, CD28, IL-23, IL-17, or IL-6 antagonist can be administered co-extensively with AFP (or a biologically active fragment, derivative, or analog thereof) and without an integrin antagonist; with an integrin antagonist and without AFP; or all three can be administered in combination.

In a second aspect, the invention features a composition that includes an AFP (or a biologically active fragment, derivative, or analog thereof) and an integrin antagonist, each in an amount (e.g., a therapeutically effective amount) to treat, prevent, or reduce one or more of the symptoms of or the progression of, MS in a patient in need thereof.

In a related embodiment, the composition further includes an amount (e.g., a therapeutically effective amount) of an antagonist of one or more of the following proteins: CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, and IL-6.

In additional embodiments of the composition, the AFP (or a biologically active fragment, derivative, or analog thereof) is in a dose in the range of 0.1 mg to 400 mg and/or the integrin antagonist is in a dose in the range of 0.1 mg to 500 mg.

The composition of the invention may be administered to a patient with MS according to the first aspect of the invention.

A third aspect of the invention features a kit that includes 1) an AFP, or a biologically active fragment, derivative, or analog thereof, and an integrin antagonist (e.g., natalizumab), each in an amount (e.g., a therapeutically effective amount) to treat, prevent, or reduce one or more of the symptoms of or the progression of, MS in a patient in need thereof, and 2) instructions for administration of the AFP and the integrin antagonist to the patient.

In additional embodiments of the kit, the AFP (or a biologically active fragment, derivative, or analog thereof) and the integrin antagonist of the kit are present in the same composition or are present in the kit in separate compositions; the separate compositions can be admixed prior to administration to a patient or they can be administered separately to the patient. In other embodiments, the AFP (or a biologically active fragment, derivative, or analog thereof) and the integrin antagonist of the kit are formulated for the same route of administration or for two different routes of administration.

In several embodiments of all the aspects of the invention, the AFP (or a biologically active fragment, derivative, or analog thereof) is recombinant human AFP having an amino acid sequence that is substantially identical (e.g., at least 60% identical, preferably at least 65%, 70%, 75%, 80%, 85%, 90%, 91%, 92%, 93%, 94%, 95%, 96%, 97%, 98%, or 99% identical, or even 100% identical) to SEQ ID NO: 1.

In other embodiments of all aspects of the invention, the AFP (or a biologically active fragment, derivative, or analog thereof) is non-glycosylated. In yet other embodiments of all aspects of the invention, the AFP may be formulated at a dosage in the range of 0.5 mg to 400 mg; the integrin antagonist may be formulated at a dosage in the range of 0.5 mg to 500 mg.

In other embodiments of all aspects of the invention, the integrin antagonist is an antibody (e.g., natalizumab), a blocking peptide, a nucleic acid inhibitor, or a small molecule inhibitor. In yet other embodiments, the integrin antagonist is an α4 integrin antagonist.

The AFP (or biologically active fragment, derivative, or analog thereof) and integrin antagonist can be formulated for or administered by one or more of a variety of routes of administration, including, but not limited to, intravenous, intramuscular, oral, by inhalation, parenteral, intraperitoneal, intraarterial, transdermal, sublingual, nasal, through use of suppositories, transbuccal, liposomal, adiposal, intraocular, subcutaneous, intrathecal, topical, or through local administration.

In additional embodiments of all aspects of the invention, the co-administration of the AFP (or a biologically active fragment, derivative, or analog thereof) and the integrin antagonist exhibits a therapeutic effect that is greater than that observed when the AFP (or biologically active fragment, derivative, or analog thereof) and the integrin antagonist are administered alone. In additional embodiments of all aspects of the invention, the AFP, the integrin antagonist, or both can be administered at a lower dosage than that normally required for achieving a therapeutic effect when either are administered alone (e.g., the AFP or the integrin antagonist can be administered at a dosage that is at least 10%, 15%, 20%, 25%, 30%, 35%, 40%, 45%, 50%, 60%, or 90% lower). In other embodiments of all aspects of the invention, co-administration of an AFP (or biologically active fragment, derivative, or analog thereof) and an integrin antagonist reduces the toxicity of the integrin antagonist (e.g., by at least 5%, 10%, 15%, 20%, 25%, 30%, 35%, 40%, 45%, 50%, 60%, 70% or more) relative to the toxicity of the integrin antagonist when administered at the same concentration in the absence of the AFP. In yet other embodiments of all aspects of the invention, the integrin antagonist can be administered in combination with the AFP at a dosage that is higher (e.g., at least 5%, 10%, 15%, 20%, 25%, 30%, 35%, 40%, 45%, 50%, 55%, 60%, 65%, 70%, 75%, 85%, 90%, 95%, 100% or more) than the normal dosage of an integrin antagonist, when administered for treating MS, without the toxicity normally expected or observed at the increased dose of the integrin antagonist when it is administered alone.

### DEFINITIONS

In this application, when therapeutic agents (e.g., an AFP and an integrin antagonist) are “administered coextensively,” the administration time periods of the agents may completely overlap or at least in part overlap. When the therapeutic agents are “administered separately,” the therapeutic agents are administered in time periods that do not overlap. In certain embodiments of separate administration, the therapeutic agents are administered in time periods that do not overlap, but are within the bioactive period for each respective agent, i.e., an earlier administered agent retains at least a substantial portion of its biological activity in the patient at the time when the latter administered agent is delivered. In other cases of separate administration, the agents are administered outside of their respective bioactive periods.

As used herein, the term “alpha-fetoprotein” or “AFP” refers to a polypeptide having an amino acid sequence that is substantially identical to the mature human AFP (SEQ ID NO: 1) or to a polypeptide that is encoded by a nucleic acid sequence that is substantially identical to the nucleic acid sequence that encodes human AFP (NCBI Accession No. NM_{—}001134; SEQ ID NO: 2). Mature human AFP is a protein of 591 amino acids (see, SEQ ID NO:1), resulting from cleavage of a precursor of 609 amino acids (GenBank Accession No. NP_{—}001125) to remove an 18-amino acid signal sequence. An AFP of this invention has an amino acid sequence that is substantially identical to SEQ ID NO: 1. The AFP is not limited to the full-length sequence; it also includes biologically active fragments of AFP. An AFP of the invention also includes any recombinant human AFP (whether or not having the same post-translational modifications as the naturally occurring version) and biologically active variants of human AFP (e.g., a non-glycosylated form of AFP; see, e.g., U.S. Pat. No. 7,208,576, incorporated by reference herein).

An AFP of this invention may contain modifications of the amino acid sequence of SEQ ID NO: 1, including substitution (e.g., conservative substitution), deletion, or addition of one or more amino acid residues. For instance, a recombinant human AFP is described in U.S. Pat. No. 7,208,576, incorporated herein by reference, which contains an asparagine to glutamine substitution at position 233 of SEQ ID NO: 1. The term “alpha-fetoprotein” also encompasses derivatives or analogs of AFP, such as those described herein.

An AFP of this invention exhibits one or more of the biological activities of the native human AFP, including, for example, the ability to bind to human leukocytes, the ability to suppress autoimmune reactions, and the ability to reduce the production of inflammatory cytokines. The leukocyte binding assay used for testing AFP activity is described herein and in, e.g., Parker et al., Protein Express. Purification 38:177-183, 2004. The autoimmune suppression activity for an AFP of this invention can be demonstrated by assaying the ability of the AFP to suppress human autologous mixed lymphocyte reactions (AMLR) or by assaying the ability of the AFP to suppress experimental autoimmune encephalomyelitis (EAE) in a mouse model using the methods described herein. The ability to reduce production of inflammatory cytokines can be assayed using the splenocyte assay described herein. A functional AFP of the invention demonstrates at least 40%, 50%, 60%, 70%, 80%, 85%, 90%, 95%, or 100% of the biological activity of the native human.

A biologically active fragment of AFP, for use in the compositions and methods of the invention, can be identified using one or more assays described herein (e.g., AMLR assays, AFP-binding to monocyte assays, experiments using the EAE mouse model, and splenocyte assays). A typical biologically active AFP fragment contains at least 5 contiguous amino acids of SEQ ID NO: 1, or at least 8 contiguous amino acids, preferably at least 10, 20, or 50 contiguous amino acids, more preferably at least 100 contiguous amino acids, and most preferably at least 200, 300, 400, or more contiguous amino acids in length. For instance, U.S. Pat. No. 6,818,741 (herein incorporated by reference) discloses an 8-amino acid fragment of human AFP (amino acids 471-478; EMTPVNPG; SEQ ID NO: 3) as well as other related AFP fragments. An active AFP fragment of this invention may further contain amino acid substitutions, deletions, or additions at a limited number of positions, so long as the AFP fragment has at least 90% identity to its corresponding sequence within SEQ ID NO: 1. For sequence comparison purposes in this application, the corresponding sequence of SEQ ID NO: 1 is deemed to have the same number of amino acids as a given AFP fragment. For instance, a 34-mer AFP peptide corresponding to the 446-479 segment of SEQ ID NO: 1 (LSEDKLLACGEGAADIIIGHLCIRHEMTPVNPGV; SEQ ID NO: 4) may contain up to 3 amino acids altered from the 446-479 segment of SEQ ID NO: 1. One such example of sequence deviation in biologically active AFP fragments is found in U.S. Pat. No. 5,707,963 (herein incorporated by reference), which discloses a 34-amino acid fragment of human AFP (SEQ ID NO: 4) with flexibility at two amino acid residues (amino acid 9 and 22 of SEQ ID NO: 4). Some other examples of AFP fragments include Domain I (amino acids 2-198 of mature human AFP; SEQ ID NO: 5), Domain II (amino acids 199-390 of mature human AFP; SEQ ID NO: 6), Domain III (amino acids 391-591 of mature human AFP; SEQ ID NO: 7), Domain I+II (amino acids 2-390 of mature human AFP; SEQ ID NO: 8), Domain II+III (amino acids 199-591 of mature human AFP; SEQ ID NO: 9), and human AFP Fragment I (amino acids 267-591 of mature human AFP; SEQ ID NO: 10).

In this application, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, and methionine methyl sulfonium. Such analogs have modified R groups (e.g., norleucine) or modified peptide backbones (e.g., peptide mimetics, such as an AFP peptoid), but retain the same basic chemical structure as a naturally occurring amino acid. Amino acid mimetics are chemical compounds that have a structure that is different from the general chemical structure of an amino acid, but that are capable of functioning in a manner that is similar to a naturally occurring amino acid. An AFP of the invention can include naturally occurring or synthetic amino acids or amino acid mimetics.

As to amino acid sequences, one of skill will recognize that individual substitutions, deletions, or additions to a polypeptide sequence that alter, add, or delete a single amino acid or a small percentage of amino acids in the sequence constitute a “conservatively modified variant,” when the alterations result in the substitution of one or more amino acids with other, chemically similar amino acids. Conservative substitution tables providing functionally similar amino acids are well known in the art. Such conservatively modified variants are in addition to and do not exclude polymorphic variants, interspecies homologs, and alleles of the invention.

The term “antibody” herein is used in the broadest sense and specifically covers monoclonal antibodies, polyclonal antibodies, multispecific antibodies (e.g., bi-specific antibodies) formed from at least two intact antibodies, and antibody fragments so long as they exhibit the desired biological activity. The term also incorporates antibody fragments (e.g., Fab, Fab′, Fv fragments, diabodies, linear antibodies, and single chain antibody molecules). The term antibody also includes monoclonal antibodies that are chimeric, primatized, or humanized, and antibody mimics.

By “antibody mimic” is meant a protein or compound that can specifically bind to a target protein (e.g., α4 integrin). Examples include molecules comprising non-immunoglobulin protein scaffolds for the variable regions of antibodies. Antibody mimics may include proteins (e.g., Adnectins), RNA molecules, unnatural oligomers (e.g., protease inhibitors, benzodiazepines, purine derivatives, and β-turn mimics).

By the term “biologically active” is meant having one or more activities known to be associated with a naturally occurring or synthetic peptide, polypeptide, protein, antibody, compound, small molecule, or fragment, derivative, or analog thereof (e.g., an AFP or fragment, derivative, or analog thereof, or an integrin antagonist).

By “blocking peptide” is meant a peptide that antagonizes the activity of a target protein by binding to the target protein and preventing its interaction with other proteins or receptors or by binding to the receptor of a target protein and blocking the interaction between the receptor and the target protein. In an embodiment, a blocking peptide is one that antagonizes the activity of α4 integrin. Examples of blocking peptides are described herein.

By “CD11/CD18” is meant a family of three heterodimeric glycoproteins with one of three α subunits (i.e., CD11a, CD11b, and CD11c) that include a sequence substantially identical to SEQ ID NO: 25 (NCBI Accession No. P20701; Larson et al., J. Cell. Biol. 108:703-712, 1989), SEQ ID NO: 26 (NCBI Accession No. P11215; Corbi et al., J. Biol. Chem. 263: 12403-12411, 1988), or SEQ ID NO: 27 (NCBI Accession No. P20702; Corbi et al., EMBO J. 6:4023-4028, 1987), or having an mRNA nucleic acid sequence that includes a nucleic acid sequence substantially identical to SEQ ID NO: 29 (NCBI Accession No. NM_{—}02209; Nishida et al., Immunity 25:583-594, 2006), SEQ ID NO: 30 (NCBI Accession No. NM_{—}000632; Arnaout et al., Proc. Natl. Acad. Sci. U.S.A. 85:2776-2780, 1988), or SEQ ID NO: 31 (NCBI Accession No. NM_{—}000887; Corbi et al., 1987, supra); and having a β subunit (CD18) which includes an amino acid sequence substantially identical to SEQ ID NO: 28 (NCBI Accession No. P05107; Kishimoto et al., Cell 48:681-690, 1987), or having a nucleic acid sequence that includes an mRNA nucleic acid sequence substantially identical to SEQ ID NO: 32 (NCBI Accession No. Y00057; Law et al., EMBO J. 6:915-919, 1987).

By “CD80” is meant a polypeptide that includes an amino acid sequence substantially identical to SEQ ID NO: 13 (NCBI Accession No. P33681; Freeman et al., J. Immunol. 143:2714-2722, 1989) or having an mRNA nucleic acid sequence comprising a nucleic acid sequence substantially identical to SEQ ID NO: 14 (NCBI Accession No. NM_{—}005191; Freeman et al., supra). CD80 is also referred to in the art as “B-lymphocyte activation antigen,” or “B7-1.”

By “dosage form” is meant the physical form of a dose of an agent of the invention (e.g., an APF and integrin antagonist). Non-limiting examples of dosage forms include a tablet, capsule, gel, cream, paste, liquid, suspension or emulsion, and spray. The dosage form can be chosen based on the intended route of administration. Alternatively, the route of administration may be dictated by the dosage form of an agent. A dosage form according to the present invention includes those that may be administered intravenously, intramuscularly, orally, parenterally, intraperitoneally, intraarterially, transdermally, sublingually, nasally, transbuccally, liposomally, adiposally, intraocularly, subcutaneously, intrathecally, topically, or through local administration.

By “hyaluronate receptor” is meant a polypeptide that includes an amino acid sequence substantially identical to SEQ ID NO: 19 (NCBI Accession No. P16070; Screaton et al., Proc. Natl. Acad. Sci. U.S.A. 89:12160-12164, 1992) or having an mRNA nucleic acid sequence that includes a nucleic acid sequence substantially identical to SEQ ID NO: 20 (NCBI Accession No. AJ251595; Gunthert, Curr. Top. Microbiol. Immunol. 184:47-63, 1993). Hyaluronate receptor is also referred to in the art as “CD44,” “phagocytic glycoprotein-1” or “PGP-1,” “lymphocyte antigen-24” or “Ly-24,” or “extracellular matrix receptor III” or “ECMR III.”

As used here, “integrin antagonist” refers to an agent that suppresses or inhibits the biological activity of an integrin molecule, such as the α4 subunit of an integrin molecule. The agent may act directly or indirectly on the α4 integrin subunit (NCBI Accession No. P13612; SEQ ID NO: 11; Takada et al., EMBO J. 8:1361-1368, 1989; or SEQ ID NO: 34) by inhibiting the activity or expression of the α4 integrin subunit, or may act on the target to which the intact integrin containing an α4 subunit binds. For example, an antibody or blocking peptide that binds to vascular cell adhesion molecule-1 (VCAM-1), thus preventing the binding of α4β1 integrin to VCAM-1 is considered an integrin antagonist for purposes of the present invention. Non-limiting exemplary integrin antagonists suitable for use with the present invention may include proteins, blocking peptides, antibodies, such as natalizumab, small molecule inhibitors, and nucleic acid inhibitors. Examples of nucleic acid inhibitors used as integrin antagonists are those that include a sequence which is complimentary to a sequence substantially identical to all or part of the mRNA sequence of human α4 integrin (NCBI Accession No. NM_{—}000885; SEQ ID NO: 12; Takada et al. supra; or SEQ ID NO: 35). For example, a sequence that is complementary to nucleotides 1-25 of SEQ ID NO: 12 or SEQ ID NO: 35.

Examples of α4 integrin antagonists include, but are not limited to, natalizumab (Elan/Biogen Idec; see, e.g., U.S. Pat. Nos. 5,840,299; 6,033,665; 6,602,503; 5,168,062; 5,385,839; and 5,730,978), oMEPUPA-V (Biogen; U.S. Pat. No. 6,495,525; incorporated by reference herein), CDP-323 (Celltech); firategrast (SB-68399; GlaxoSmithKline); TR-9109 (Pfizer); ISIS-107248 (Antisense Therapeutics); R-1295 (Roche); and TBC-4746 (Schering-Plough).

Additional non-limiting examples of α4 integrin antagonists include the small molecules described in U.S. Pat. Nos. 5,821,231; 5,869,448; 5,936,065; 6,265,572; 6,288,267; 6,365,619; 6,423,728; 6,426,348; 6,458,844; 6,479,666; 6,482,849; 6,596,752; 6,667,331; 6,668,527; 6,685,617; 6,903,128; and 7,015,216 (each herein incorporated by reference); in U.S. Patent Application Publication Nos. 2002/0049236; 2003/0004196; 2003/0018016; 2003/0078249; 2003/0083267; 2003/0100585; 2004/0039040; 2004/0053907; 2004/0087574; 2004/0102496; 2004/0132809; 2004/0229858; 2006/0014966; 2006/0030553; 2006/0166866; 2006/0166961; 2006/0241132; 2007/0054909; and 2007/0232601 (each herein incorporated by reference); in European Patent Nos. EP 0842943; EP 0842944; EP 0842945; EP 0903353; and EP 0918059; and in PCT Publication Nos. WO 95/15973; WO 96/06108; WO 96/40781; WO 98/04247; WO 98/04913; WO 98/42656; WO 98/53814; WO 98/53817; WO 98/53818; WO 98/54207; WO 98/58902; WO 99/06390; WO 99/06431; WO 99/06432; WO 99/06433; WO 99/06434; WO 99/06435; WO 99/06436; WO 99/06437; WO 99/10312; WO 99/10313; WO 99/20272; WO 99/23063; WO 99/24398; WO 99/25685; WO 99/26615; WO 99/26921; WO 99/26922; WO 99/26923; WO 99/35163; WO 99/36393; WO 99/37605; WO 99/37618; WO 99/43642; WO 01/42215; and WO 02/28830; all of which are incorporated by reference herein.

Additional examples of α4 integrin antagonists include the phenylalanine derivatives described in: U.S. Pat. Nos. 6,197,794; 6,229,011; 6,329,372; 6,388,084; 6,348,463; 6,362,204; 6,380,387; 6,445,550; 6,806,365; 6,835,738; 6,855,706; 6,872,719; 6,878,718; 6,911,451; 6,916,933; 7,105,520; 7,153,963; 7,160,874; 7,193,108; 7,250,516; and 7,291,645 (each herein incorporated by reference). Additional amino acid derivatives that are α4 integrin antagonists include those described in, e.g., U.S. Patent Application Publication Nos. 2004/0229859 and 2006/0211630 (herein incorporated by reference), and PCT Publication Nos. WO 01/36376; WO 01/47868; and WO 01/70670; all of which are incorporated by reference herein.

Other examples of α4 integrin antagonists include the peptides, and the peptide and semi-peptide compounds described in, e.g., PCT Publication Nos. WO 94/15958; WO 95/15973; WO 96/00581; WO 96/06108; WO 96/22966 (Leu-Asp-Val tripeptide; Biogen, Inc.); WO 97/02289; WO 97/03094; and WO 97/49731. An additional example of an α4 integrin antagonist is the pegylated molecule described in U.S. Patent Application Publication No. 2007/066533 (herein incorporated by reference).

Examples of antibodies that are α4 integrin antagonists include those described in, e.g., PCT Publication Nos. WO 93/13798; WO 93/15764; WO 94/16094; and WO 95/19790. Additional examples of α4 integrin antagonists are described herein.

By “leukocyte function antigen-1” or “LFA-1” is meant a polypeptide that includes an α subunit having an amino acid sequence substantially identical to SEQ ID NO: 21 (NCBI Accession No. P20701; Larsen et al., J. Cell. Biol. 108:703-712, 1989) and a β subunit having an amino acid sequence substantially identical to SEQ ID NO: 22 (NCBI Accession No. P05107; Kishimoto et al., Cell 48:681-690, 1987); or having an mRNA nucleic acid sequence that includes a nucleic acid sequence substantially identical to SEQ ID NO: 23 (NCBI Accession No. Y00796; Larsen et al., supra) or SEQ ID NO: 24 (NCBI Accession No. Y00057; Law et al., EMBO J. 6:915-919, 1987).

By “nucleic acid inhibitor” is meant any nucleic acid sequence (DNA or RNA) or peptide-nucleic acid sequence that contains a sequence complimentary to a nucleic acid sequence that is substantially identical to all or part of the mRNA of a targeted protein (e.g., for α4 integrin, a sequence complimentary to SEQ ID NO: 12 or SEQ ID NO: 35; for CD80, a sequence complimentary to SEQ ID NO: 14; for P-selectin, a sequence complimentary to SEQ ID NO: 16; for sphingosine-1-phosphate receptor-1, a sequence complimentary to SEQ ID NO: 18; for hyaluronate receptor, a sequence complimentary to SEQ ID NO: 20; for LFA-1, a sequence complimentary to SEQ ID NOS: 23 or 24; and for CD11/CD18, a sequence complimentary to SEQ ID NOS: 29, 30, 31, or 32), which, when administered to a cell or subject, results in decreased activity or expression of the target protein (e.g., α4 integrin, CD80, P-selectin, sphingosine-1-phosphate receptor-1, hyaluronate receptor, LFA-1, CD11/18, CD20, CD86, ICOS ligand, CCR2, CXCR3, CCR5, CD40, CD154, CD28, IL-23, IL-17, or IL-6.) relative to a cell or subject not administered the nucleic acid inhibitor. Examples of nucleic acid inhibitors include RNAi, antisense RNA, siRNA, miRNA, and peptide-nucleic acids. One example of a nucleic acid inhibitor is a nucleic acid sequence that results in a decrease in the activity or expression of α4 integrin.

Examples of antisense nucleic acid molecules include RNA or DNA molecules having a sequence that is complementary to the mRNA nucleic acid sequences of target proteins; the sequence of an antisense nucleic acid molecule of the invention has a length of at least 10, 20, 25, 30, 40, 50, 60, 70, 80, 90, or 100, or 100 or more nucleotides. Other examples of antisense nucleic acid molecules are interfering RNA or RNAi molecules (e.g., small interfering RNAs or siRNAs). RNAi molecules contain sequences that are complementary to the mRNA nucleic acid sequences of target proteins (as described above), and may include heterologous sequences which facilitate hairpin formation. RNAi molecules may be at least 10, 20, 25, 30, 40, 50, or even greater than 50 nucleotides in length; preferably the RNAi molecule is 21 or 25 nucleotides in length. Typically, RNAi molecules have at least 25 nucleotides and are complementary to any 25 nucleotides of the target protein mRNA sequence (e.g., the mRNA sequence of α4 integrin set forth in SEQ ID NO: 12 or SEQ ID NO: 35). Additional examples of RNAi molecules are those that are complementary to nucleotides 1-25, 50-75, 50-100, 50-150, 50-1000, or 50-2000 of SEQ ID NO: 12 or SEQ ID NO: 35.

By “P-selectin” is meant a polypeptide having an amino acid sequence substantially identical to SEQ ID NO: 15 (NCBI Accession No. NP_{—}002966; Johnston et al., Cell 56:1033-1044, 1989) or having an mRNA nucleic acid sequence that includes a nucleic acid sequence substantially identical to SEQ ID NO: 16 (NCBI Accession No. NM_{—}003005; Johnston et al., supra). P-selectin is also referred to in the art as “CD62P,” “Granule Membrane Protein-140” or “GMP-140,” and “Platelet Activation-Dependent Granule to External Membrane Protein” or “PADGEM.”

By “small molecule inhibitor” is meant any small molecule which is identified as an antagonist of a target protein (e.g., α4 integrin, CD80, P-selectin, sphingosine-1-phosphate receptor-1, hyaluronate receptor, LFA-1, CD11/18, CD20, CD86, ICOS ligand, CCR2, CXCR3, CCR5, CD40, CD154, CD28, IL-23, IL-17, or IL-6) using screening or biological assays (e.g., ligand binding assays, protein or receptor activity assays, and other assays as known in the art). The compound may be identified by screening commercially available chemical or small molecule libraries.

By “sphingosine-1-phosphate receptor-1” or “S1P1” is meant a polypeptide having an amino acid sequence substantially identical to SEQ ID NO: 17 (NCBI Accession No. P21453; Hla and Maciag, J. Biol. Chem. 265:9308-9313, 1990) or having an mRNA nucleic acid sequence that includes a nucleic acid sequence substantially identical to SEQ ID NO: 18 (NCBI Accession No. BC018650; Strausberg et al., supra). Sphingosine-1-phosphate receptor-1 is also referred to in the art as “endothelial differentiation, sphingolipid G-protein coupled receptor-1” or “Edg1.”

The term “substantial identity” or “substantially identical,” when used in the context of comparing a polynucleotide or polypeptide sequence to a reference sequence, refers to the fact that the polynucleotide or polypeptide sequence is the same as the reference sequence or has a specified percentage of nucleotides or amino acid residues that are the same at the corresponding locations within the reference sequence when the two sequences are optimally aligned. For instance, an amino acid sequence that is “substantially identical” to a reference sequence has at least about 60% identity, preferably at least 65%, 70%, 75%, 80%, 85%, 90%, 91%, 92%, 93%, 94%, 95%, 96%, 97%, 98%, or 99% identity, or higher percentage identity (up to 100%) to a reference sequence (e.g., the mature human AFP amino acid sequence as set forth in SEQ ID NO: 1, the human AFP mRNA nucleic acid sequence set forth in SEQ ID NO: 2, or a pre-determined segment of SEQ ID NOS: 1 or 2), when compared and aligned for maximum correspondence over the full length of the reference sequence as measured using BLAST or BLAST 2.0 sequence comparison algorithms with default parameters, or by manual alignment and visual inspection (see, e.g., NCBI web site).

A “therapeutically effective amount” of a therapeutic agent (e.g., an AFP or an integrin antagonist) is an amount of the agent that is sufficient to treat or reduce one or more symptoms of MS (e.g., tingling, numbness, tremors, loss of balance, weakness in one or more limbs, blurred or double vision, slurred speech, swallowing problems, paralysis, lack of coordination, cognitive difficulties, fatigue, muscle spasms, dizziness, breathing problems, or seizures) or the severity of one or more symptoms of MS (e.g., by at least about 5%, 10%, 15%, 20%, 25%, 30%, 40%, 50%, 60%, 70%, or 80% relative to an untreated control patient). The treatment of MS or reduction in one or more of the symptoms of MS (or their severity) can be determined by using one of several methods known in the art (see, e.g., the Expanded Disability Status Scale (EDSS), Kurtzke, Neurology 33:1444-1452, 1983; and the Multiple Sclerosis Severity Score (MSSS), Roxburgh et al., Neurology 64:1144-1151, 2005). Such amount may vary depending on the effect to be achieved. For instance, a “therapeutically effective amount” of an integrin antagonist for treating MS when used in combination with AFP (or a biologically fragment thereof) may be different from the “therapeutically effective amount” of an integrin antagonist when used alone for treating MS. In different embodiments, the therapeutic effect is to reduce the symptoms (e.g., muscle weakness and demylination of nerves) or progression of MS in a patient.

By “treating” is meant the reduction (e.g., by at least 10%, 15%, 20%, 25%, 30%, 35%, 40%, 45%, 50%, 60%, 70%, 80%, 90%, 95%, 99%, or even 100%) in the progression, severity, or frequency of one or more symptoms of MS (e.g., tingling, numbness, tremors, loss of balance, weakness in one or more limbs, blurred or double vision, slurred speech, swallowing problems, paralysis, lack of coordination, cognitive difficulties (e.g., decreased memory and concentration), fatigue, muscle spasms, dizziness, breathing problems, and seizures) or the prevention or decrease (e.g., by at least 10%, 15%, 20%, 25%, 30%, 35%, 40%, 45%, 50%, 60%, 70%, 80%, 90%, 95%, 99%, or even 100%) in the progression of MS in a human patient (e.g., demyelination of nerves and the frequency or severity of one or more symptoms of MS).

### BRIEF DESCRIPTION OF THE FIGURES

FIG. 1 shows the amino acid sequence of mature human AFP (SEQ ID NO: 1) and the mRNA nucleic acid sequence of human AFP (SEQ ID NO: 2). N indicates the asparagine 233 glycosylation site in the mature human AFP amino acid sequence.

FIG. 2 shows the amino acid sequences of biologically active fragments of AFP including amino acids 2-198 (Domain I; SEQ ID NO: 5), amino acids 199-390 (Domain II; SEQ ID NO: 6), amino acids 391-591 (Domain III; SEQ ID NO: 7), amino acids 2-390 (Domains I+II; SEQ ID NO: 8), amino acids 199-591 (Domains II+III; SEQ ID NO: 9), and amino acids 261-591 of mature human AFP (Human AFP Fragment 1; SEQ ID NO: 10).

FIG. 3 shows two different amino acid sequences for α4 integrin protein (SEQ ID NOS: 11 and 34) and two different nucleic acid sequences for α4 integrin mRNA (SEQ ID NOS: 12 and 35).

FIG. 4 shows the amino acid sequence (SEQ ID NO: 13) and the nucleic acid sequence (SEQ ID NO: 14) of CD80 protein and mRNA, respectively.

FIG. 5 shows the amino acid sequence (SEQ ID NO: 15) and the nucleic acid sequence (SEQ ID NO: 16) of P-selectin protein and mRNA, respectively.

FIG. 6 shows the amino acid sequence (SEQ ID NO: 17) and the nucleic acid sequence (SEQ ID NO: 18) of sphingosine-1-phosphate receptor-1 protein and mRNA, respectively.

FIG. 7 shows the amino acid sequence (SEQ ID NO: 19) and the nucleic acid sequence (SEQ ID NO: 20) of hyaluronate receptor protein and mRNA, respectively.

FIG. 8 shows the amino acid sequence (SEQ ID NO: 21) and the nucleic acid sequence (SEQ ID NO: 23) of LFA-1 α subunit protein and mRNA, respectively; and the amino acid sequence (SEQ ID NO: 22) and the nucleic acid sequence (SEQ ID NO: 24) of LFA-1 β subunit protein and mRNA, respectively.

FIG. 9 shows the amino acid sequences of CD11a (SEQ ID NO: 25), CD11b (SEQ ID NO: 26), CD11c (SEQ ID NO: 27), and CD18 (SEQ ID NO: 28); and the nucleic acid sequences of CD11a (SEQ ID NO: 29), CD11b (SEQ ID NO: 30), CD11c (SEQ ID NO: 31), and CD18 (SEQ ID NO: 32) mRNA.

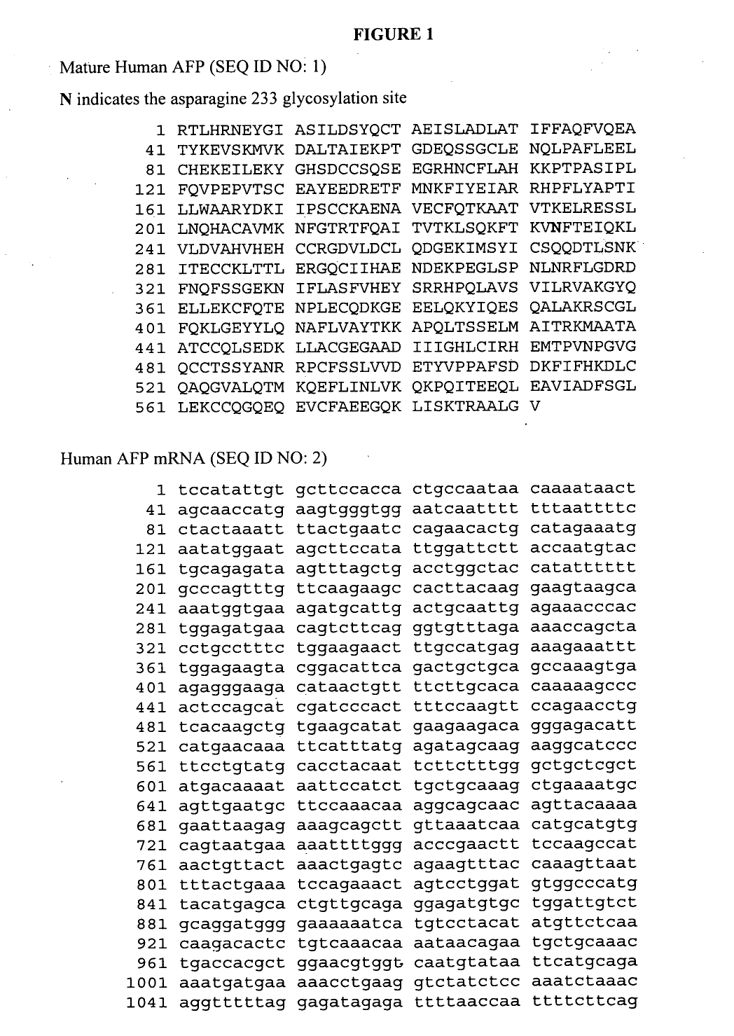

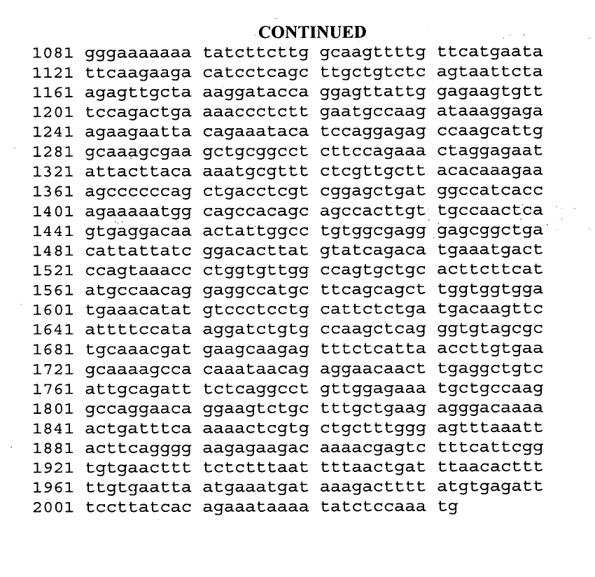

### DETAILED DESCRIPTION

The invention features a combination therapy for treating MS that involves the co-administration of an integrin antagonist and an AFP (or a biologically active fragment thereof), each in a therapeutically effective amount, to an MS patient in need thereof.

The invention also features a pharmaceutical composition that includes both an integrin antagonist and an AFP (or a biologically active fragment thereof), each in a therapeutically effective amount for treating, preventing, or reducing one or more of the symptoms of or the progression of, MS. Such a composition optionally contains one or more pharmaceutically acceptable excipients and is formulated to be administered intravenously, intramuscularly, orally, by inhalation, parenterally, intraperitoneally, intraarterially, transdermally, sublingually, nasally, through use of suppositories, transbuccally, liposomally, adiposally, intraocularly, subcutaneously, intrathecally, topically, or through local administration.

The invention also features a kit for treating, preventing, or reducing one or more of the symptoms of or the progression of, MS, which includes a therapeutically effective amount of an integrin antagonist and AFP (or a biologically active fragment thereof), along with proper instructions for using the kit.

The integrin antagonist may be an antibody, a blocking peptide, a nucleic acid inhibitor, or a small molecule inhibitor. Examples of integrin antagonists are described herein. The AFP can be full length AFP, a biologically active fragment, derivative, or analog thereof, or a mutein thereof having one or more amino acid substitutions, deletions, or additions. Examples of AFP agents of the invention are described herein. The AFP and the integrin antagonist may be formulated for or administered in a single dosage form or they may be formulated or administered in different dosage forms. The AFP and integrin antagonist of the invention can be administered coextensively or separately. In addition, the AFP or integrin antagonist may be administered one or more times hourly, daily, weekly, biweekly, or monthly.

An antagonist to CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6 may also be administered in the methods and compositions of the invention.

### Diagnosis and Monitoring of Multiple Sclerosis

MS can be diagnosed by observing one or more symptoms in a patient. Symptoms of MS may be single or multiple and may range from mild to severe in intensity and short to long in duration. Complete or partial remission from symptoms occurs early in about 70% of MS patients. Visual disturbances often are the first symptoms of MS, but they usually subside. A patient may notice blurred or double vision, red-green distortion, or sudden blindness. Muscle weakness leading to difficulties with coordination and balance commonly is noticed early. Muscle spasms, fatigue, numbness, and prickling pain are common symptoms. There may be a loss of sensation, speech impediment, tremors, dizziness, or occasionally hearing loss. Fifty percent of patients experience mental changes such as decreased concentration, attention deficits, some degree of memory loss, or impairment in judgment. Other symptoms may include depression, manic depression, paranoia, or an uncontrollable urge to laugh and weep called laughing-weeping syndrome. As the disease worsens, patients may experience sexual dysfunction or reduced bowel and bladder control. Heat appears to intensify MS symptoms for about 60% of patients, and relief is found with cold baths or swimming. Pregnancy seems to reduce the number of attacks.

There is no single test for MS. Physicians, particularly neurologists, can take into consideration detailed medical histories and can perform complete physical and neurological examinations in order to diagnose MS. Testing for MS can include, e.g., magnetic resonance imaging (MRI) with intravenous gadolinium or magnetic resonance scanning (MRS), both of which help to identify, describe, and date lesions in the brain (i.e., plaques) that occur in MS patients. Another electro-physiological test, evoked potentials, examines the impulses traveling through the nerves to determine if the impulses are moving normally or too slowly; slower than normal movement of impulses through the nerves is indicative of MS. Finally, examination of the cerebro-spinal fluid that surrounds the spinal cord may be used to identify abnormal chemicals or cells floating in the brain or spinal cord that suggest the presence of MS. Collectively, these three tests strengthen the diagnosis of MS. MS can also be diagnosed by identifying one or more of the following symptoms in a patient: tingling, numbness, tremors, loss of balance, weakness in one or more limbs, blurred or double vision, slurred speech, swallowing problems, paralysis, lack of coordination, cognitive difficulties (e.g., decreased memory and concentration), fatigue, muscle spasms, dizziness, breathing problems, and seizures.

All of the methodologies described above are also useful for monitoring the progression of MS in patients, as well as for monitoring the resolution of MS following treatment using the compositions and methods of the invention (e.g., the resolution or decrease in the severity or frequency of one or more symptoms of MS), such that the effectiveness of the treatment received by the patient can be assessed. In addition, a patient can be assessed for an improvement in MS following treatment (e.g., an improvement in one or more symptoms of MS (e.g., a decrease in the occurrence, length, or severity of one or more of the symptoms of MS), or for an improvement in motor neural function) using one of several methods known in the art (see, e.g., the Expanded Disability Status Scale (EDSS), Kurtzke, Neurology 33:1444-1452, 1983; and the Multiple Sclerosis Severity Score (MSSS), Roxburgh et al., Neurology 64:1144-1151, 2005).

### Methods of Treatment of MS by Administration of the Compositions of the Present Invention

The present invention features methods of treating MS in a patient by co-administering a therapeutically effective amount of an AFP (or biologically active fragment thereof) and an integrin antagonist; the compositions of the invention may, but need not, also include additional therapeutic agents, such as those described below. The compositions of the invention can be administered to a patient to treat, prevent, ameliorate, inhibit the progression of, or reduce the severity of one or more symptoms of MS in a human patient. The AFP (or biologically active fragment thereof) and an integrin antagonist may be administered coextensively or separately, in a single dose or in multiple doses. The AFP (or biologically active fragment thereof) and integrin antagonist may be formulated for the same route of administration or formulated for different routes of administration.

Examples of the symptoms of MS that can be treated using the compositions of the invention include: tingling, numbness, tremors, loss of balance, weakness in one or more limbs, blurred or double vision, slurred speech, swallowing problems, paralysis, lack of coordination, cognitive difficulties (e.g., decreased memory and concentration), fatigue, muscle spasms, dizziness, breathing problems, and seizures. These symptoms of MS, and their resolution during treatment, may be measured by a physician during a physical examination or by using one or more of the tests described above.

A physician may adjust the dose (e.g., increase or decrease the dose) of the AFP or of the integrin antagonist administered to the patient based on the severity of, occurrence of, or progression of MS in the patient. For example, a physician can increase the dose of the AFP or of the integrin antagonist if necessary to alleviate one or more symptoms of MS in a patient. Alternatively, a physician can decrease the dose of the AFP or of the integrin antagonist based on an improvement in one or more symptoms of MS in the patient or to avoid toxicity associated with, e.g., the administration of an integrin antagonist.

The combination therapies of the present invention, which include, e.g., an AFP and an integrin antagonist, preferably exhibit a greater therapeutic effect (e.g., improved efficacy or reduced toxicity at higher doses) than that observed when either agent is administered alone.

### COMPOSITIONS OF THE PRESENT INVENTION

The present invention provides compositions including an AFP (or a biologically active fragment, derivative, or analog thereof) and an integrin antagonist (e.g., an α4 integrin antagonist) for the treatment of MS. The compositions of the invention may be formulated for any route of administration (e.g., the formulations described herein) and may be administered in a single dose or in multiple doses to a subject in need thereof. The compositions of the invention may also include supplemental agents, e.g., a CD80, P-selectin, sphingosine-1-phosphate receptor-1, hyaluronate receptor, leukocyte function antigen-1 (LFA-1), CD11/CD18, CD20, CD86, ICOS ligand, CCR2, CXCR3, CCR5, CD40, CD154, CD28, IL-23, IL-17, or IL-6 antagonist. The supplemental agents can be antibodies, blocking peptides, nucleic acid inhibitors, or small molecule inhibitors that antagonist CD80, P-selectin, sphingosine-1-phosphate receptor-1, hyaluronate receptor, leukocyte function antigen-1 (LFA-1), CD11/CD18, CD20, CD86, ICOS ligand, CCR2, CXCR3, CCR5, CD40, CD154, CD28, IL-23, IL-17, or IL-6.

### AFPs for Use in the Compositions and Methods of the Invention

An alpha-fetoprotein for use in the compositions and methods of the present invention are described below. For the purposes of the present invention, both naturally occurring human AFP and recombinantly produced AFP polypeptides or biologically active fragments thereof can be used. Naturally occurring human AFP can be obtained by, e.g., purification from umbilical cord blood or umbilical cord serum. Recombinant AFP polypeptide or biologically active fragment thereof can be obtained, e.g., by using a prokaryotic or eukaryotic expression system, such as those described in, e.g., U.S. Pat. No. 5,384,250 and U.S Patent Application Publication No. 20040098755 (each of which is herein incorporated by reference). These methods include the purification of AFP from a biological fluid of transgenic mammal that has been engineered to express AFP into the biological fluid, as well as other methods known in the art. These AFPs can be used in the present invention notwithstanding the fact that the use of these different expression systems (e.g., production in a prokaryotic host cell, a eukaryotic host cell, or a transgenic animal or plant) may result in a recombinant AFP or fragment thereof having different post-translational modifications than that in the wild-type AFP (e.g., a different number of attached sugar residues (e.g., at least 1, 2, 3, 4, 5, 6, 7, or 10 sugar molecules), a different type of glycosylation (e.g., O-linked glycosylation or N-linked glycosylation, or the lack thereof), a different type of sugar residues (e.g., mannose, galactose, N-acetyl-galactosamine, N-acetyl-glucosamine, glucuronate, sialic acid, or xylose, or different combinations thereof), or different amino acids glycosylated). For instance, naturally occurring human AFP is a variably glycosylated protein (e.g., glycosylation at asparagine 233 of SEQ ID NO: 1). In contrast, the recombinant AFP or fragment may be unglycosylated when produced by a prokaryotic host cell or may be somewhat differently glycosylated when produced by a eukaryotic host cell. Alternatively, a recombinant AFP can be genetically modified to eliminate glycosylation (e.g., by removing a glycosylation site, for instance replacing asparagine 233 of SEQ ID NO: 1 with any amino acid other than asparagine), regardless of the expression system in which it is produced. Human AFP is available through various commercial suppliers, including Fitzgerald Industries International (Concord, Mass.), Cell Sciences (Canton, Mass.), and Biodesign International (Saco, Me.).

Furthermore, it is possible to employ well-known chemical synthesis methods to synthesize an AFP polypeptide or fragment, particularly when the AFP fragment is a peptide of a relatively short length, e.g., with less than 50 or 100 amino acids.

Any AFP polypeptide or fragment thereof, regardless of its origin or status of post-translational modification, can be used in the present invention if the polypeptide or fragment has the same or substantially the same biological activity (e.g., at least about 40%, desirably at least about 50%, 60%, 70%, and more desirably at least about 80%, 90%, 100%, or 100% or more of the biological activity) of native human AFP (e.g., as determined based on the ability of the AFP to bind to human leukocytes, to suppress human autologous mixed lymphocyte reactions (AMLR), to suppress EAE in a mouse model, or to inhibit release of inflammatory cytokines in a splenocyte assay).

Similarly, fragments of the human AFP can also be used in the compositions and methods of the present invention, so long as the fragments retain the same or substantially the same biological activity of naturally occurring human AFP (as determined using one or more of the assays for AFP biological activity described below). Fragments of human AFP can be generated by methods known to those skilled in the art, e.g., proteolytic cleavage or recombinant expression, or may result from normal protein processing (e.g., removal from a nascent polypeptide of amino acids that are not required for biological activity). Fragments of human AFP can also be produced recombinantly using the techniques described above. Chemical methods can also be useful for synthesizing active AFP fragments.

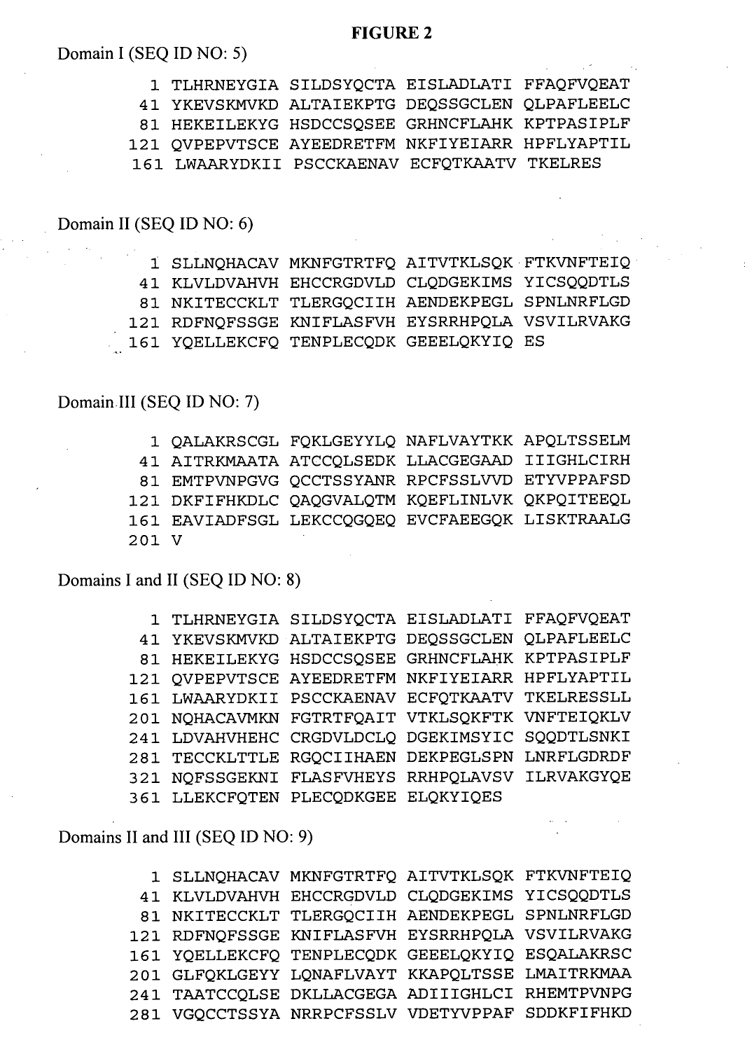

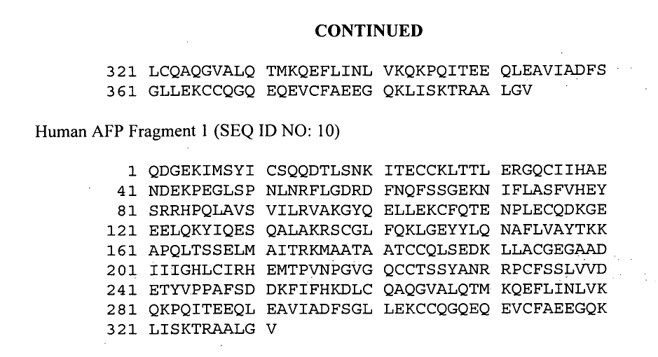

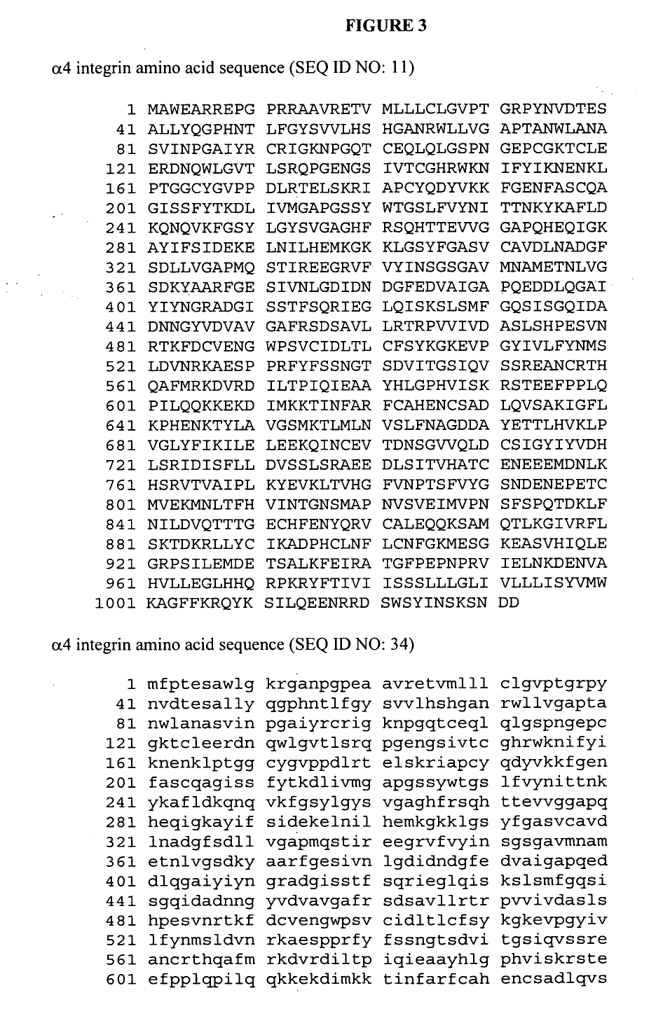

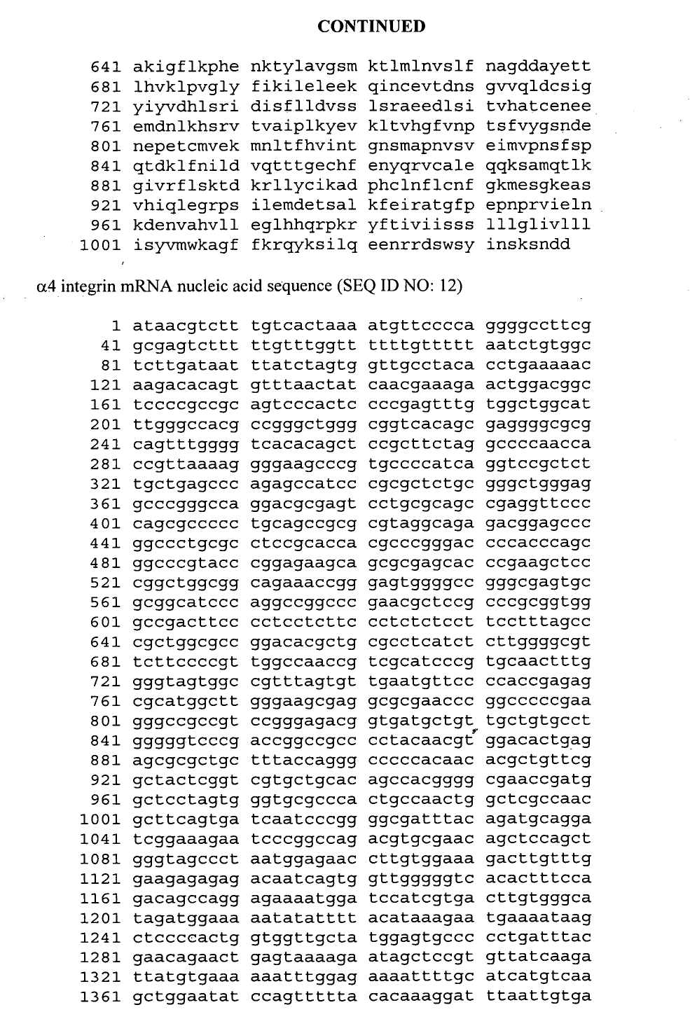

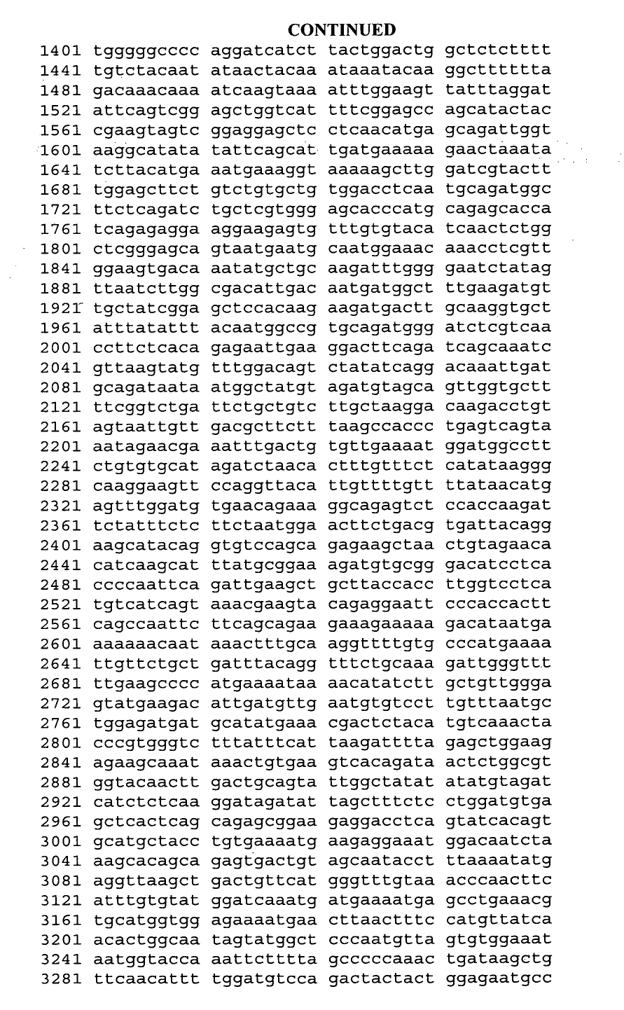

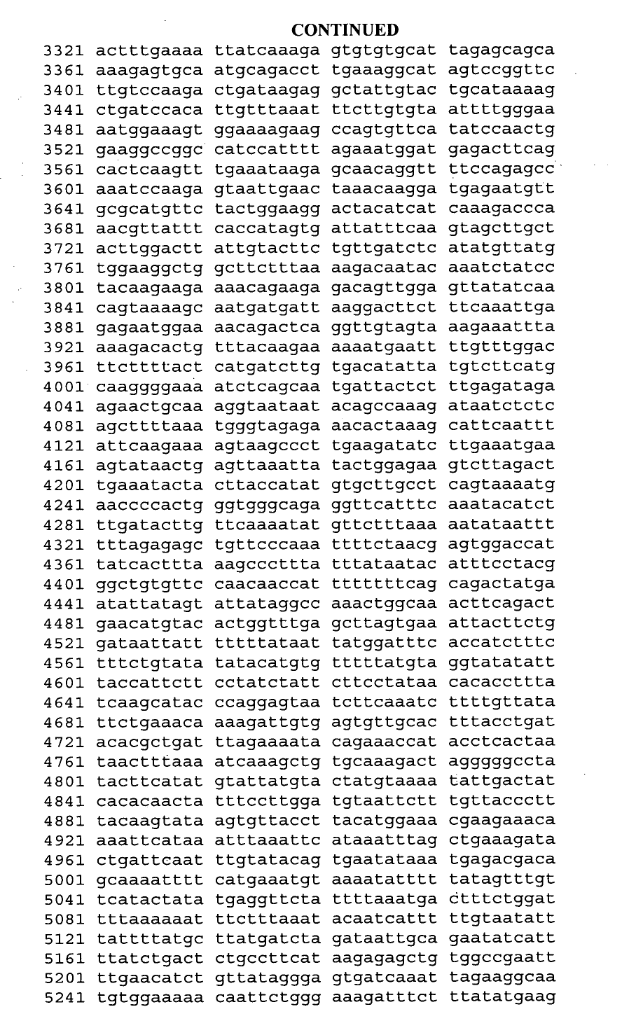

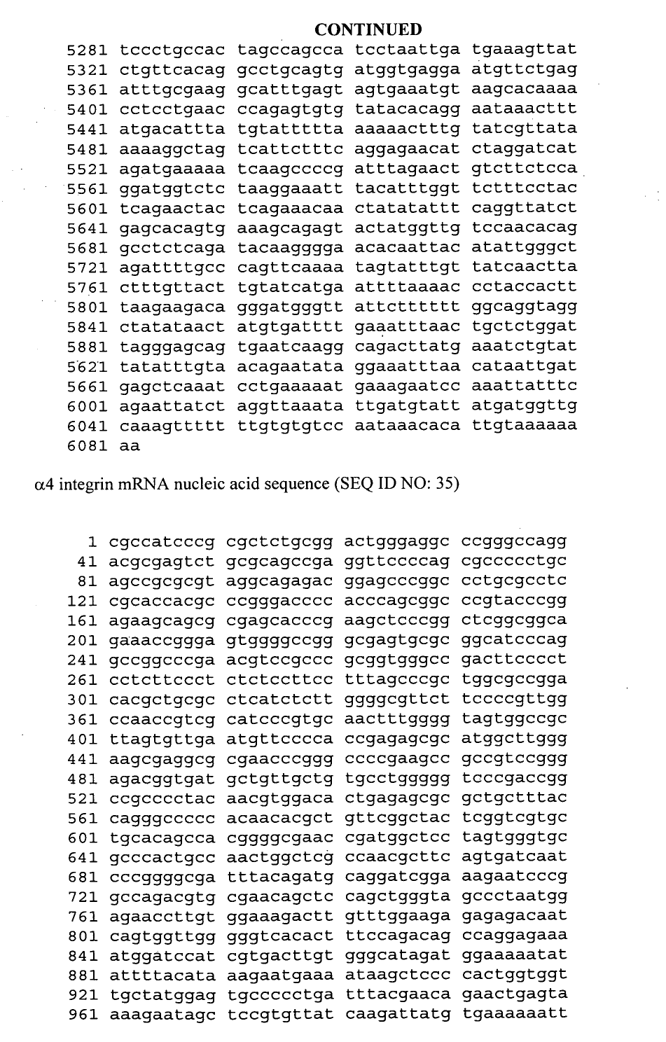

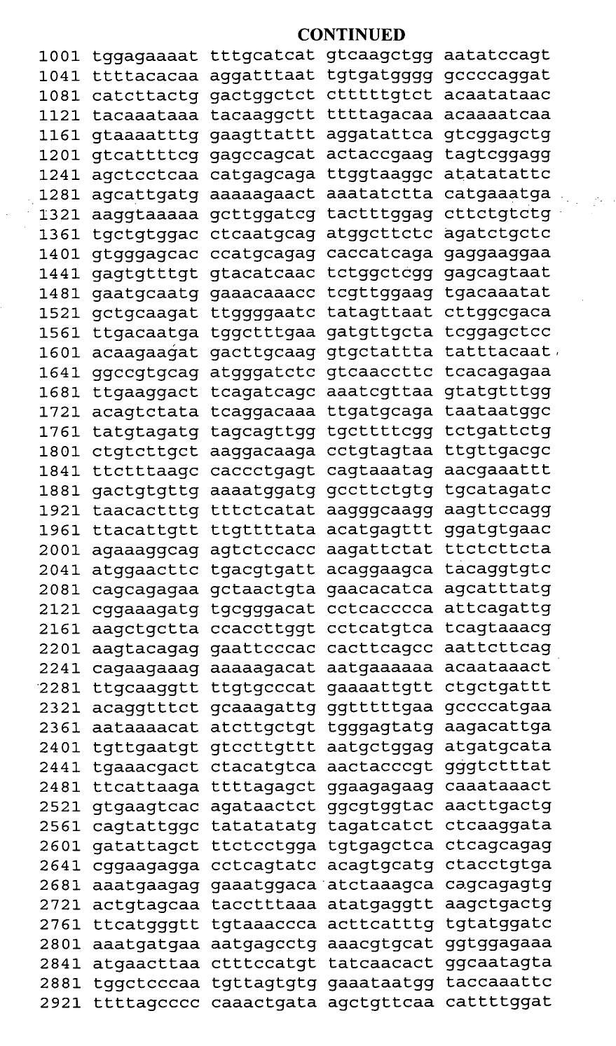

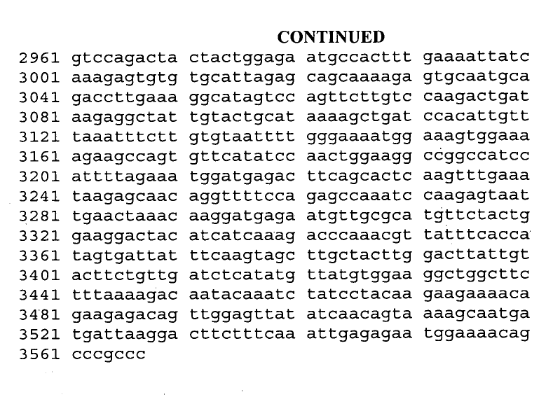

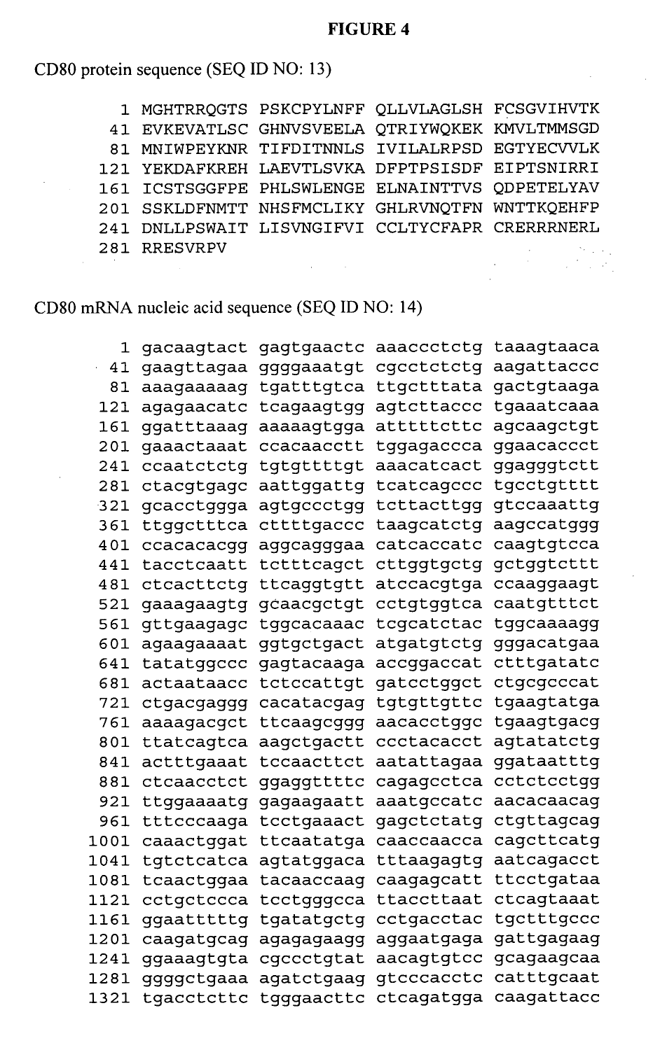

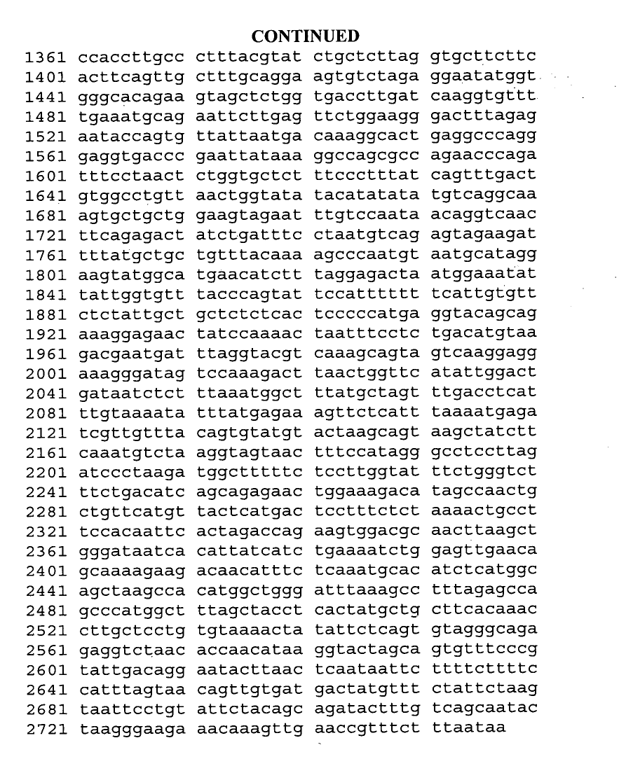

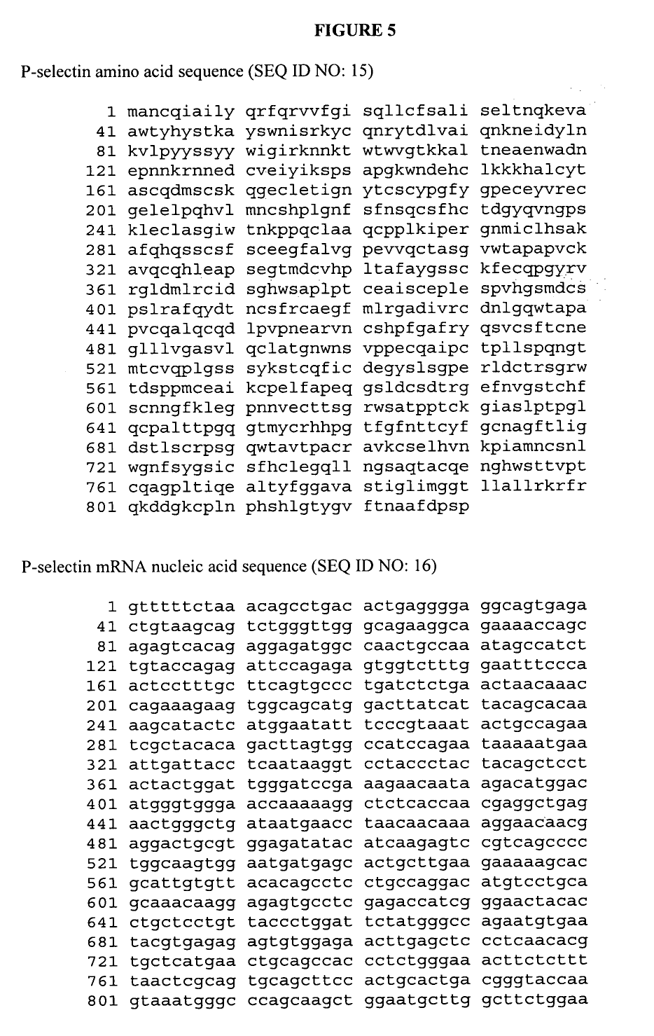

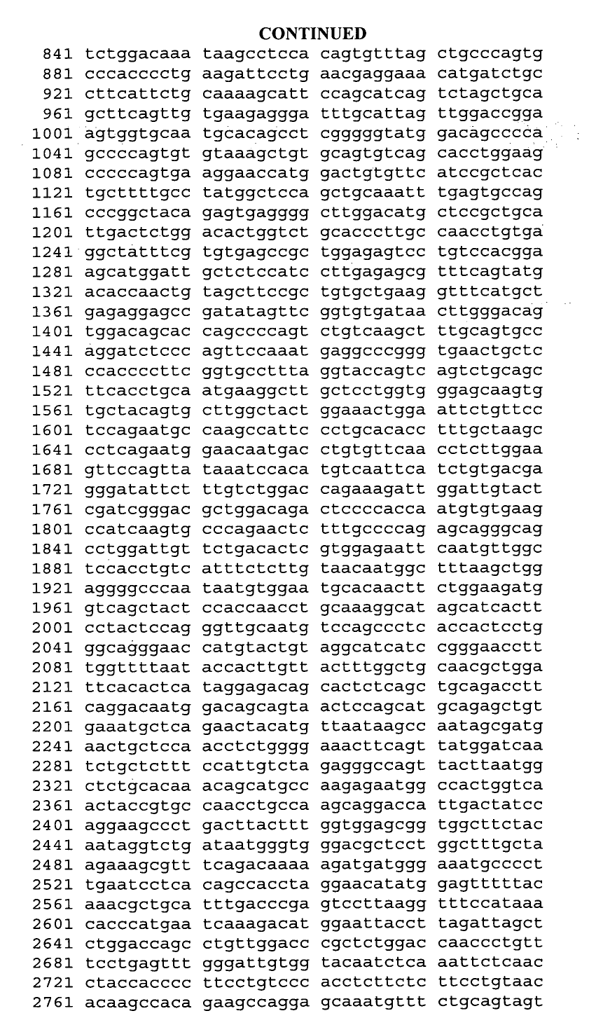

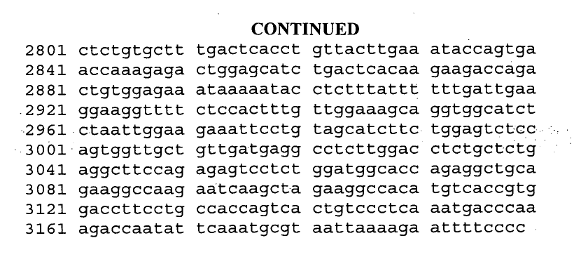

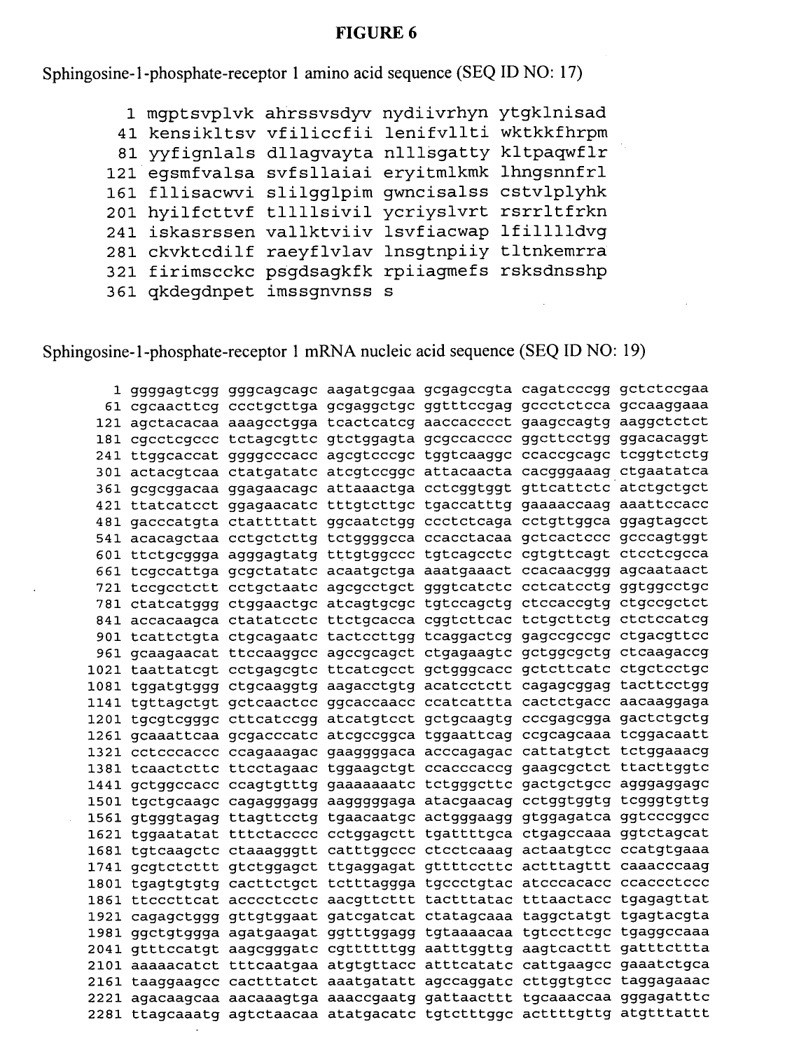

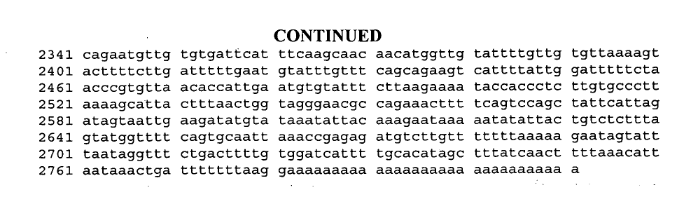

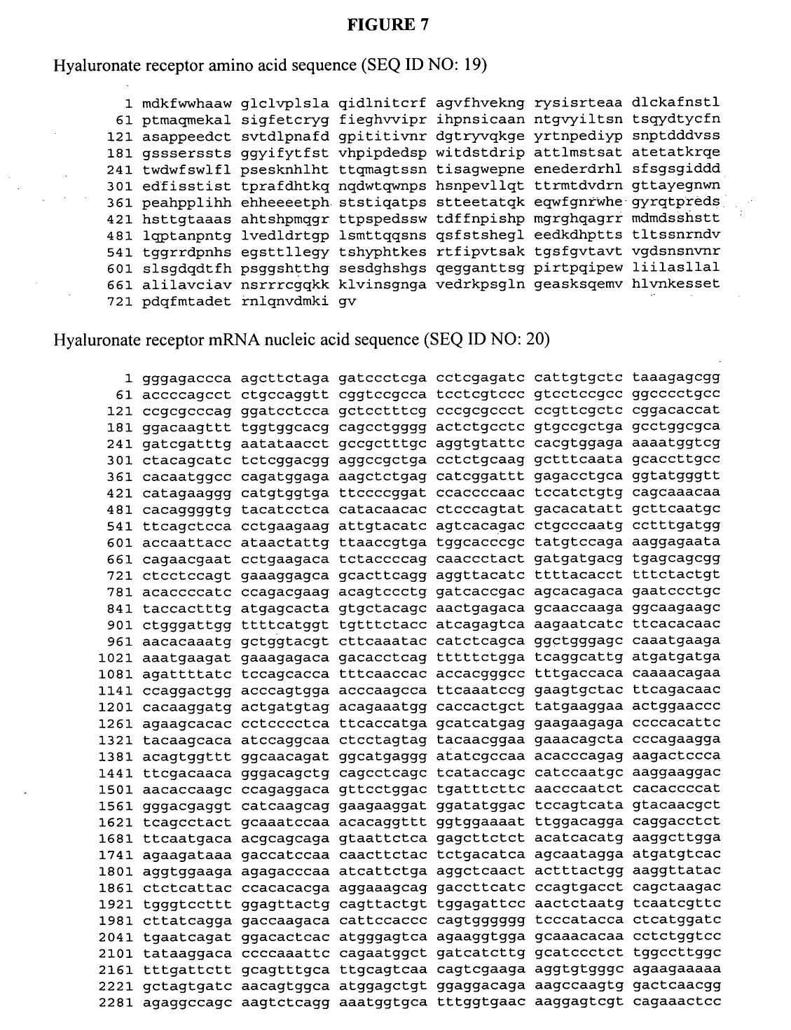

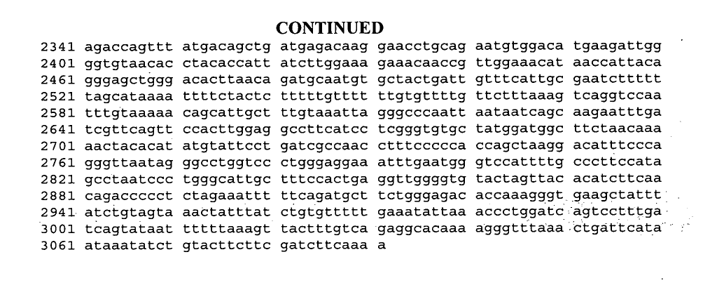

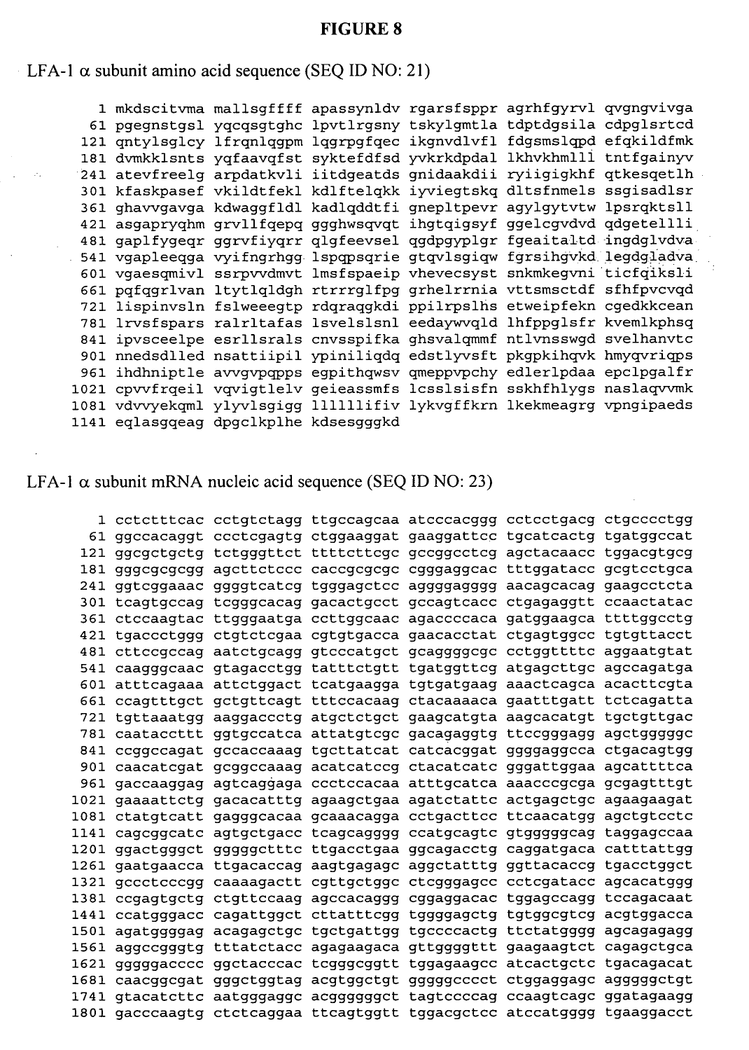

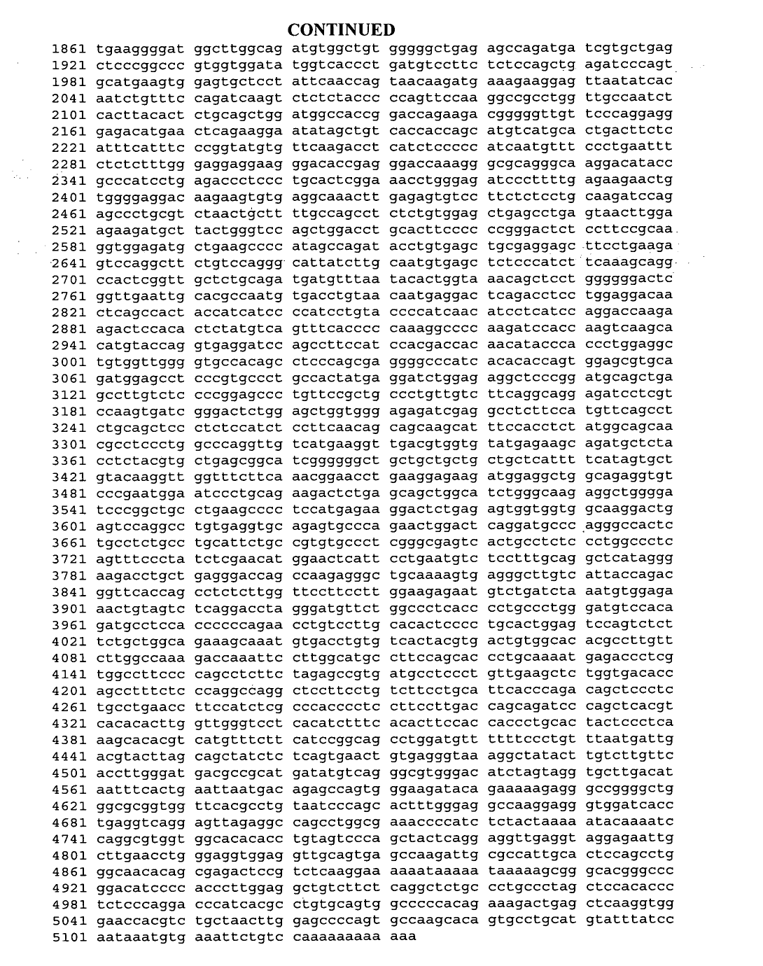

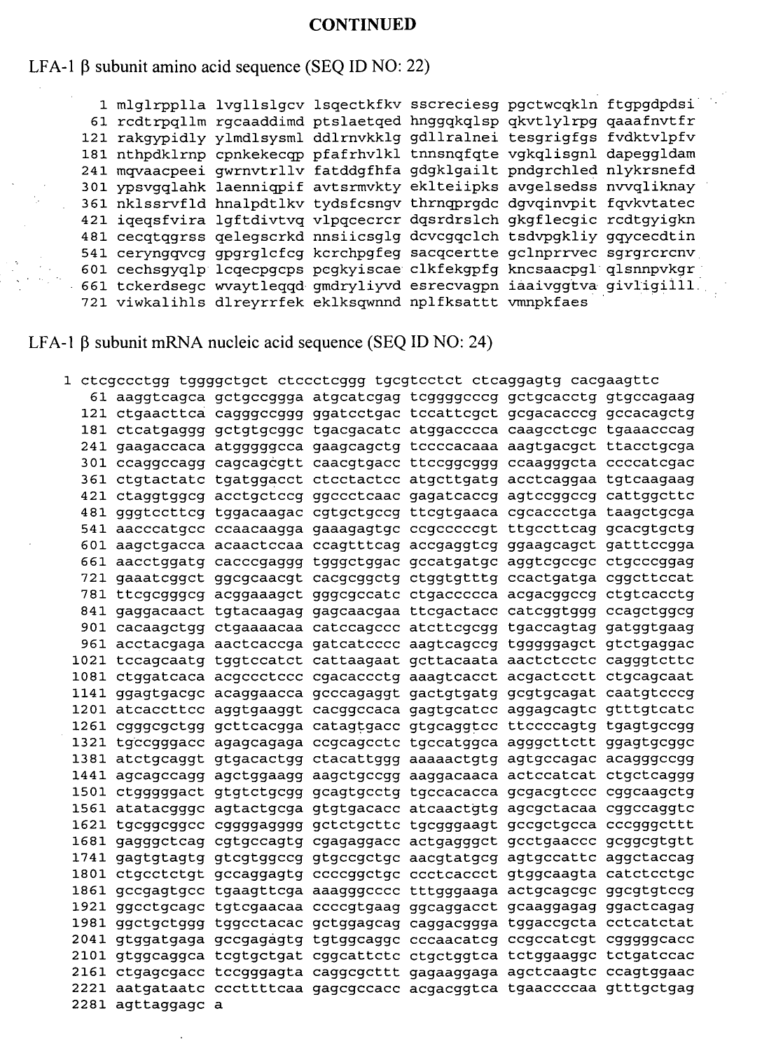

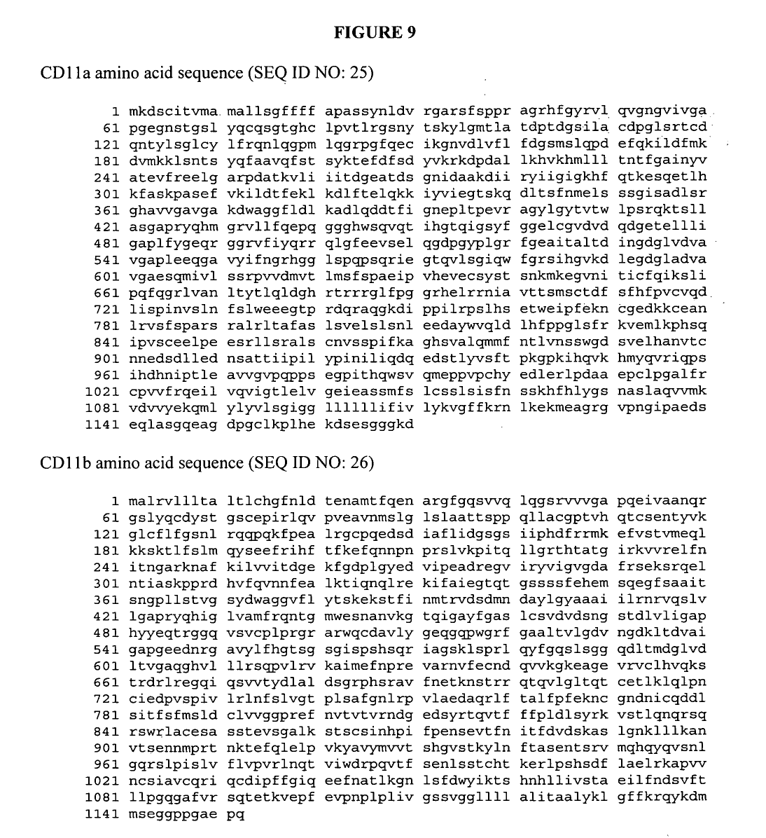

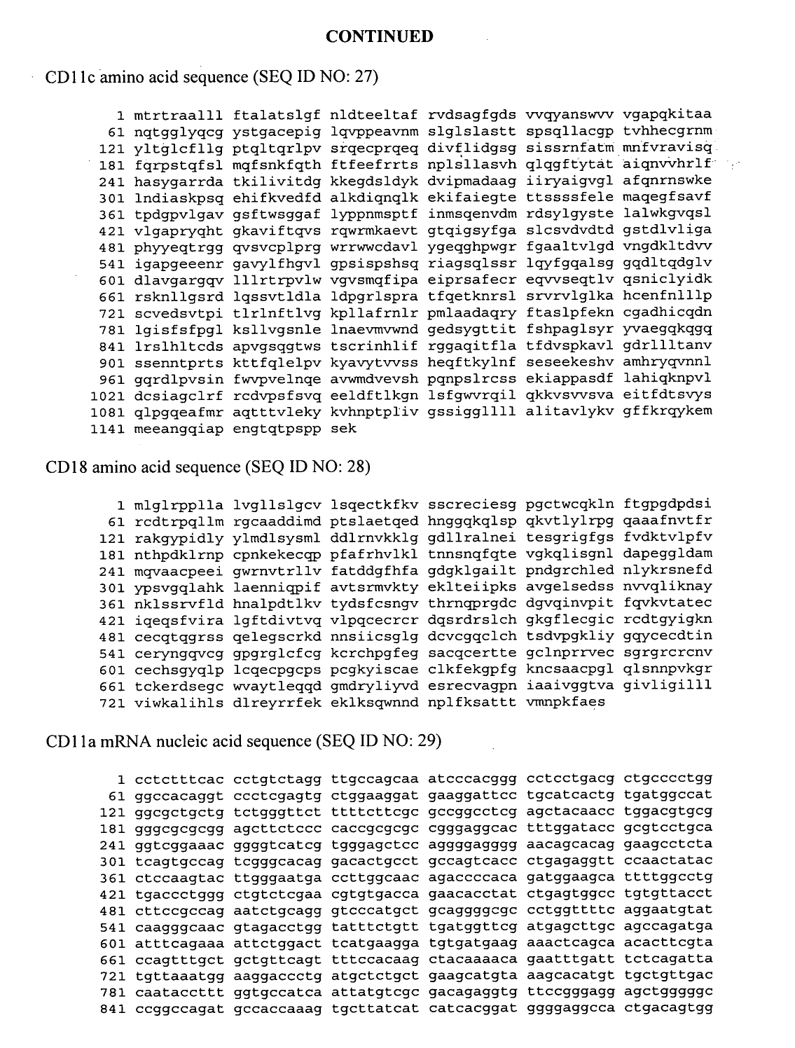

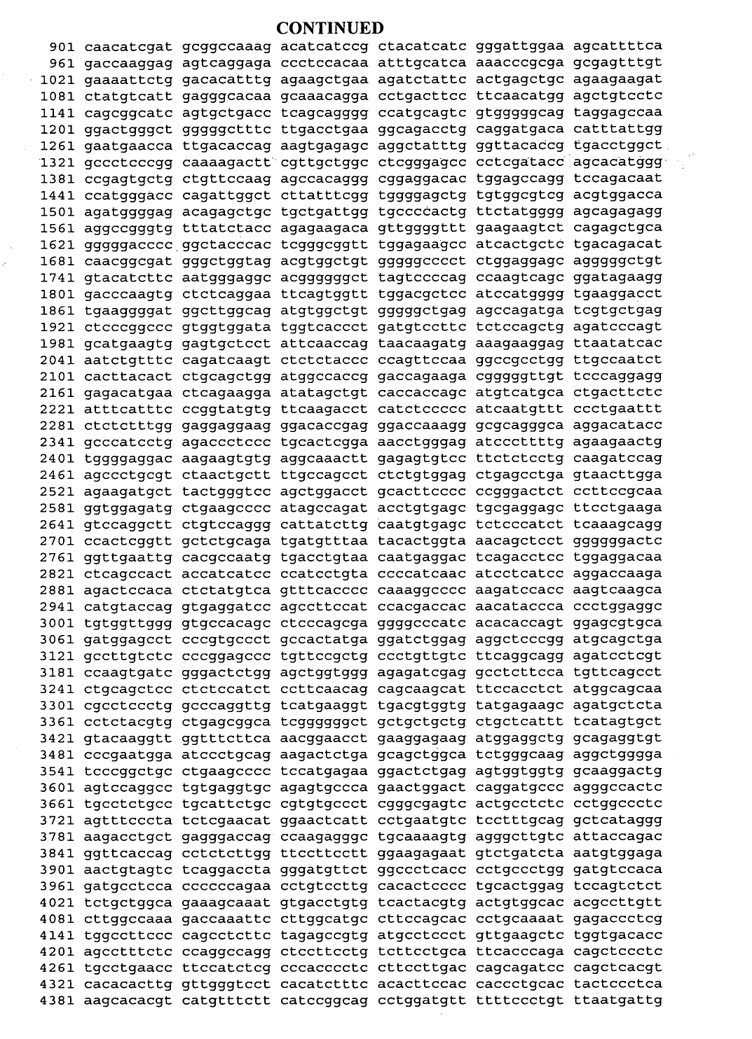

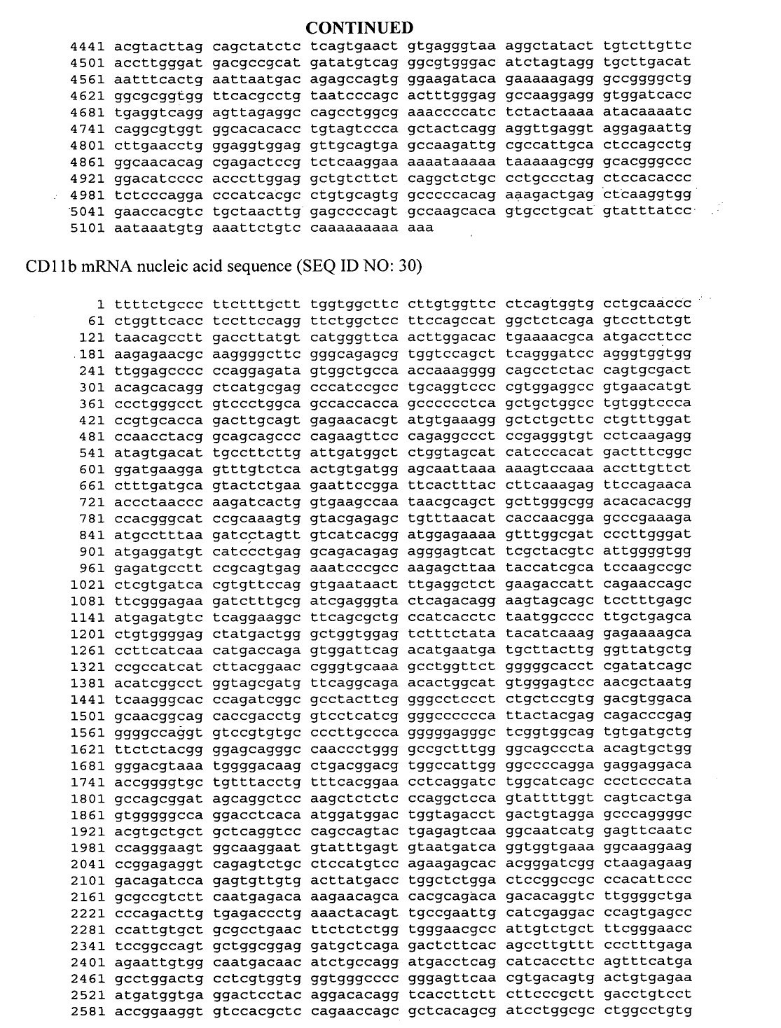

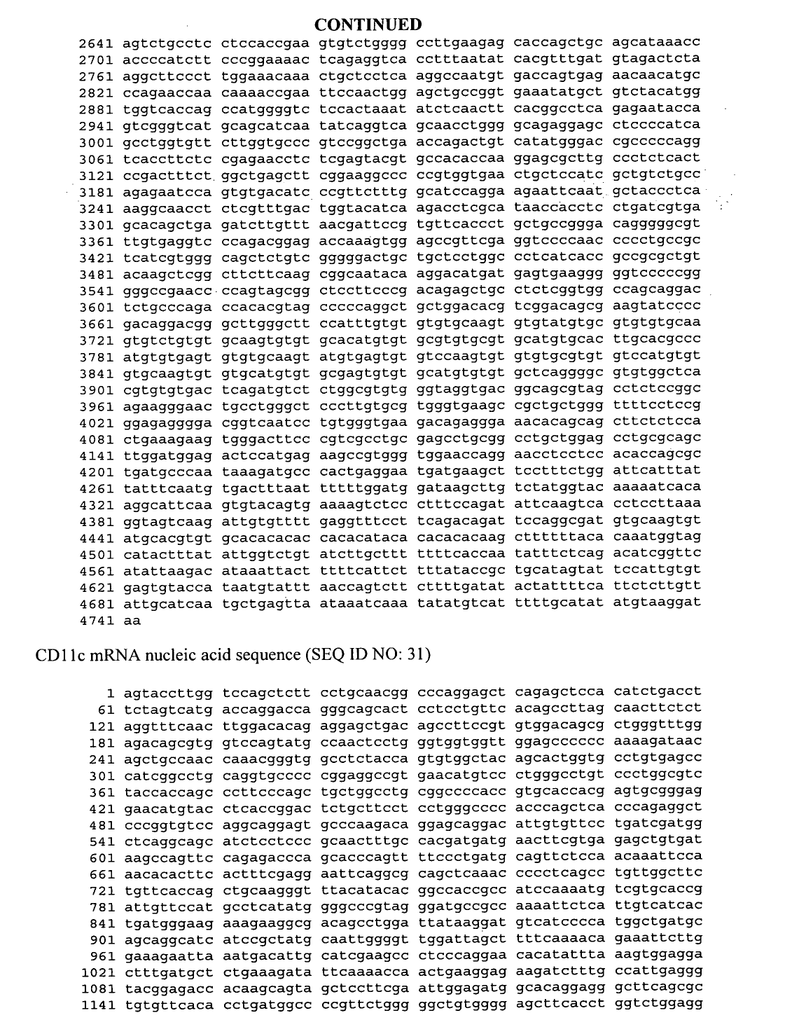

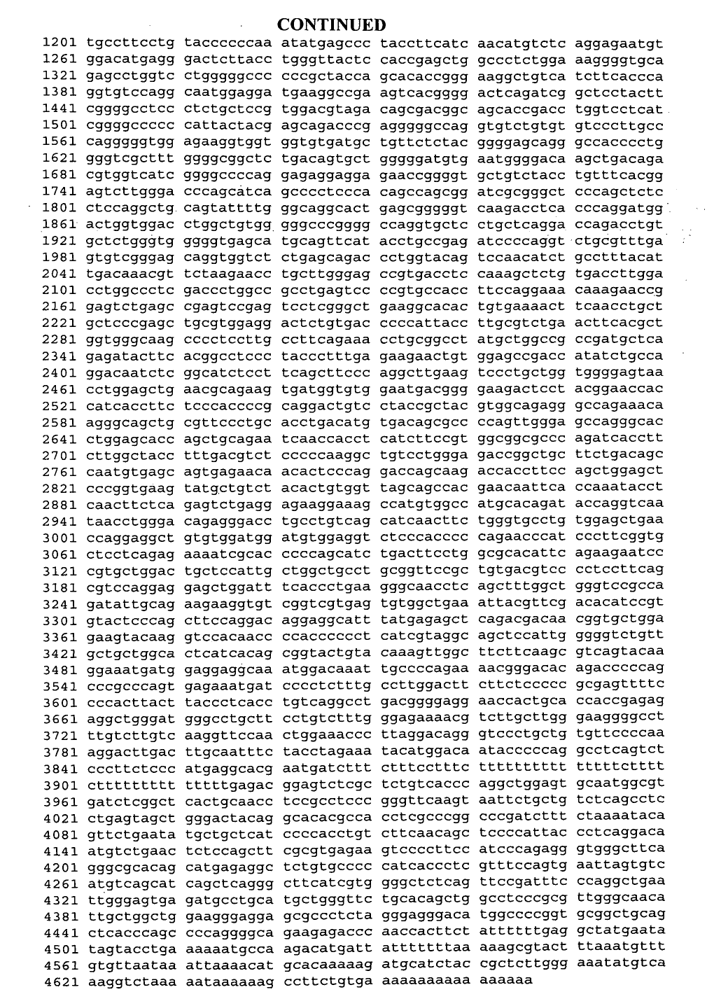

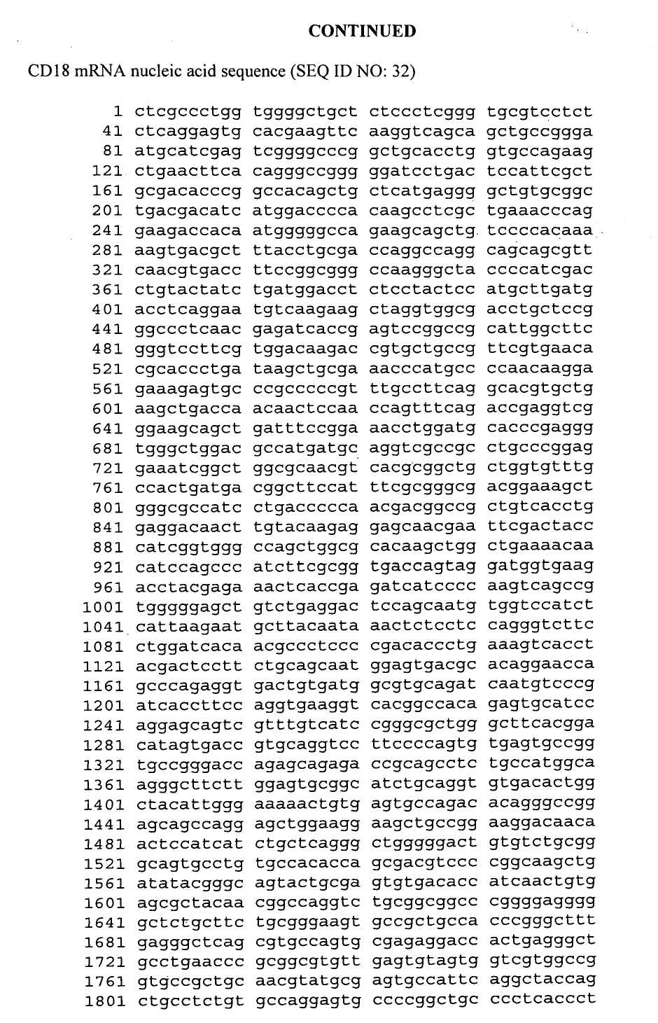

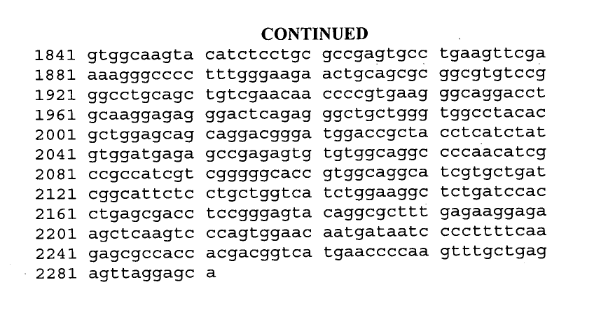

Examples of human AFP fragments suitable for use in practicing the present invention are shown in FIG. 2 and include Domain I (amino acids 2-198 of mature human AFP; SEQ ID NO: 5), Domain II (amino acids 199-390 of mature human AFP; SEQ ID NO: 6), Domain III (amino acids 391-591 of mature human AFP; SEQ ID NO: 7), Domain I+II (amino acids 2-390 of mature human AFP; SEQ ID NO: 8), Domain II+III (amino acids 199-591 of mature human AFP; SEQ ID NO: 9), and AFP Fragment I (amino acids 267-591 of mature human AFP; SEQ ID NO: 10). Other examples of known AFP fragments are described herein or can be found in, e.g., U.S. Pat. No. 5,707,963 and U.S. Pat. No. 6,818,741, herein incorporated by reference.

Also encompassed within the claimed invention is the use of functional derivatives or analogs of full-length native human AFP or fragments thereof. As described in earlier sections, such derivatives or analogs can differ from the full-length native human AFP or portions thereof by amino acid sequence differences (e.g., additions, deletions, conservative or non-conservative substitutions), or by modifications (e.g., post-translational modifications) that do not affect sequence, or by both. The derivatives/analogs of the invention will generally exhibit at least 90%, more preferably at least 95%, or even 99% amino acid identity with all or part of the native human AFP amino acid sequence (SEQ ID NO: 1).

An AFP derivative/analog may differ from a naturally occurring human AFP due to post-translational modifications (which do not normally alter primary sequence), which include in vivo, or in vitro chemical derivatization of polypeptides, e.g., acetylation, carboxylation, or pegylation; such modifications may occur during polypeptide synthesis or processing, or following treatment with isolated modifying enzymes. Also included are cyclized peptide molecules and analogs that contain residues other than L-amino acids, e.g., D-amino acids, non-naturally occurring, or synthetic amino acids, e.g., β or γ amino acids, or L-amino acids with non-natural side chains (see, e.g., Noren et al., Science 244:182, 1989). Methods for site-specific incorporation of non-natural amino acids into the protein backbone of proteins is described, e.g., in Ellman et al., Science 255:197, 1992. Also included are chemically synthesized polypeptides or peptides with modified peptide bonds (e.g., non-peptide bonds as described in U.S. Pat. No. 4,897,445 and U.S. Pat. No. 5,059,653; herein incorporated by reference) or modified side chains to obtain the desired pharmaceutical properties as described herein. Useful AFP, AFP fragments, AFP derivatives, and AFP analogs having the same or substantially the same biological activity (e.g., at least about 40%, desirably at least about 50%, 60%, 70%, and more desirably at least about 80%, 90%, 100%, or 100% or more of the biological activity) of wild-type AFP can be identified using art-recognized methods, such as those described below.

Some preferred functional AFP derivatives contain one or more conservative substitutions, in which certain amino acid residues are substituted by other residues having similar chemical structures (e.g., alanine for glycine, arginine for lysine, etc.). The derivatives/analogs mentioned above may include allelic variants, inter-species variants, and genetic variants, both natural and induced (for example, resulting from random mutagenesis by, e.g., site-specific mutagenesis according to methods described in scientific literature such as Sambrook et al., Molecular Cloning, A Laboratory Manual, 3rd ed., 2001; Kriegler, Gene Transfer and Expression: A Laboratory Manual, 1990; and Ausubel et al., eds., Current Protocols in Molecular Biology, 1994.

### AFP Activity Assays

As stated above, AFP polypeptides or AFP fragments suitable for use in the compositions and methods of the present invention may include various derivatives, analogs, or fragments of the naturally occurring human AFP, so long as the polypeptides or fragments retain the same or substantially the same biological activity of mature human AFP. The biological activity of an AFP of the present invention can be demonstrated by using, e.g., one or more of the following assays.

A first assay for testing a candidate AFP for biological activity entails measuring the ability of the AFP to specifically bind to human leukocytes (e.g., peripheral monocytes). A binding assay suitable for this purpose is described in, e.g., Parker et al., Protein Express. Purification 38:177-183, 2004. Briefly, a competitive assay format is used to test a candidate AFP for its ability to specifically bind to U937 cells, a human monocytic cell line. The cells are maintained in RPMI media with 10% fetal bovine serum. Prior to the binding assay, the cells are washed twice with serum-free media and adjusted to 2.5×10⁶ cells/ml in phosphate-buffered saline (PBS). Native human AFP (SEQ ID NO: 1) or non-glycosylated human AFP (see, e.g., SEQ ID NO: 36, where, e.g., residue 233 is glutamine) is labeled with a detectable label, e.g., fluorescein, in a proper reaction followed by removal of the unattached labeling material, for instance, by gel filtration. In the case of labeling human AFP with fluorescein, the protein is mixed with a solution of fluorescein-5-isothiocyanate in dimethyl sulfoxide for 1 hour in the dark, followed by gel filtration to remove unbound dye. Labeled human AFP is stored in 20% glycerol at −20° C. until use.

For the binding assay, a certain number of U937 cells (e.g., 40 μl of cell suspension at 2.5×10⁶ cells/ml concentration) are mixed with a pre-determined amount of labeled human AFP (e.g., at a final concentration of 0.5 μM) and with unlabeled human AFP or an unlabeled candidate AFP, each at a set of final concentrations (e.g., 20, 10, 5, 2.5, 1.25, and 0.625 μM) to determine the IC₅₀ values for both human AFP and the candidate AFP. At the conclusion of the binding process, the cells are then washed with PBS and suspended in fresh PBS so that the labeled AFP remaining on U937 cells can be measured, e.g., by flow cytometry.

A second assay for testing a candidate AFP for biological activity entails measuring the ability of the AFP to suppress autoimmune reactions, either in AMLR or in a mouse model of EAE. Methods are known in the art for testing AMLR and its inhibition. For instance, U.S. Pat. Nos. 5,965,528 and 6,288,034 (each of which is herein incorporated by reference) describe the AMLR system as follows: isolation of human peripheral blood mononuclear cells (PBMC), their fractionation into non-T-cell populations, and the AMLR, performed according to standard procedures. Briefly, responder T-cells are isolated by passing 1.5×10⁸ PMBC over a commercial anti-Ig affinity column (US Biotek Laboratories, Seattle, Wash.) and 2×10⁵ responder cells are subsequently cultured with 2×10⁵ autologous ¹³⁷Cs-irradiated (2500 rads) non-T stimulator cells from a single donor. The medium employed consists of RPMI-1640 supplemented with 20 mM HEPES (Invitrogen), 5×10⁻⁵ M 2-mercaptoethanol (BDH, Montreal, QC), 4 mM L-glutamine (Invitrogen), 100 U/ml penicillin (Invitrogen), and 100 μg/ml streptomycin sulfate, with the addition of 10% fresh human serum autologous to the responder T-cell donor for the AMLR. Varying concentrations of purified recombinant human AFP, human serum albumin, anti-human AFP monoclonal antibody clone #164 (125 μg/ml final concentration in culture) (Leinco Technologies, St. Louis, Mo.) are added at the initiation of cultures. AMLR cultures are incubated for 4 to 7 days, at 37° C. in 95% air and 5% CO₂. At the indicated intervals, DNA synthesis is assayed by a 6 hour pulse with 1 μCi of ³H-thymidine (specific activity 56 to 80 Ci/mmole; ICN Radioisotopes, Cambridge, Mass.). The cultures are harvested on a multiple sample harvester (Skatron, Sterling, Va.), and the incorporation of ³H-TdR is measured in a Packard 2500 TR liquid scintillation counter. Results are expressed as mean cpm±the standard error of the mean of triplicate or quadruplicate cultures.

The immunosuppressive activity of a candidate AFP within the scope of the present invention can be assessed by its ability to suppress human autologous mixed lymphocyte reactions (AMLR). Generally, the candidate AFP is tested for its ability to inhibit the proliferative response of autoreactive lymphocytes stimulated by autologous non-T-cells, by measuring lymphocyte autoproliferation throughout a time course of 4 to 7 days. Suppression of AMLR in a dose-dependent manner is demonstrated by results from dose-response studies performed at the peak of T-cell autoproliferation where an AFP is added at the initiation of cultures. Furthermore, parallel viability studies can be used to establish that the inhibitory activity of an AFP polypeptide or fragment on human autoreactive T-cells is not due to non-specific cytotoxic effects.

A third assay for testing a candidate AFP for biological activity involves the use of a myelin oligodendrocyte glycoprotein (MOG) mouse model of experimental autoimmune encephalomyelitis (EAE) (see, e.g., Fritz et al., J. Immunol. 130:1024, 1983; Naiki et al., Int. J. Immunopharmacol. 13:235, 1991; and Goverman, Lab. Anim. Sci., 46:482, 1996). In this in vivo assay, genetically susceptible strains of mice are subcutaneously immunized with MOG emulsified in Complete Freund's Adjuvant (CFA), which leads to the development of EAE in the animals. A candidate AFP is administered to a selected group of mice on a daily basis, beginning prior to, at the same time, or subsequent to the start of the administration of MOG to the animals. The symptoms of EAE in these animals are monitored and compared to those in a control group (e.g., those receiving only saline injections) over a certain time period, e.g., 30 days. Severity of EAE in each animal is given a score between 1-5 based on defined clinical symptoms; the average score of animals in a group indicates the disease state of the group. A biologically active AFP will reduce the severity of EAE in animals receiving MOG compared to controls (e.g., at least a 50% reduction in the severity of disease after 30 days of treatment).

A fourth assay that can be used to test a candidate AFP for biological activity examines the ability of the candidate AFP to inhibit or reduce the release of inflammatory cytokines from mitogen-stimulated in vitro splenocyte cultures obtained from naïve mice (e.g., as described in Hooper and Evans, J. Reprod. Immunol. 16: 83-961, 1989; and Kruisbeek, in Current Protocols in Immunology, Vol. 1, Section 3.1.1-3.1.5, 2000). Splenocytes are stimulated with phytohemagglutinin (PHA), concavalin A (ConA), or lipopolysaccharide (LPS) in the presence of increasing concentrations of an AFP for 24 hours. Human serum albumin is used as a negative control for the assays. A 10 point dose response study has shown that biologically active AFP inhibits or substantially inhibits the secretion of PHA induced IFN-γ in a reproducible manner.

### Integrin Antagonists for Use in the Compositions and Methods of the Invention

An integrin antagonist for use in the methods of this invention is an agent that decreases (e.g., by at least 5%, 10%, 15%, 20%, 25%, 30%, 35%, 40%, 45%, 50%, 60%, 70%, 80% or more) the biological activity or expression (transcription or translation) of α4 integrin. Integrin antagonists can include antibodies, blocking peptides, nucleic acid inhibitors, or small molecule inhibitors. As understood in the field of biological science, integrins are integral membrane proteins present in the plasma membrane of cells, that, inter alia, play a role in signal transduction and in the attachment of a cell to the extracellular matrix (ECM). There are many types of integrins, and many cells have multiple types on their surface.

Structurally, integrins are non-covalent heterodimeric complexes consisting of an α subunit and a β subunit. See, Hemeler, Ann. Rev. Immunol. 8:365, 1990. About 18α and 8 β subunits have been characterized, with additional variants formed due to differential splicing. Through the different combinations of α and β subunits, at least 24 unique integrins can be generated.

The α4β1 integrin, or very late antigen-4 (VLA-4), is constituitively expressed by all leukocytes (e.g., monocytes, lymphocytes, basophils, eosinophils, mast cells, macrophages, and neutrophils). The binding of VLA-4 to one of its ligands has a number of known cell adhesion and activation functions. See, for example, Hemeler, supra; Walsh et al., Clin. and Exp. Allergy, 25:1128, 1995; and Hutala et al., J. Cell Biol. 129:867, 1995. In particular, VLA-4 interacts with the cytokine inducible endothelial cell surface protein known as vascular cell adhesion molecule-1 (VCAM-1), and the alternatively spliced forms of the extracellular matrix protein fibronectin containing the CS-1 domain (Ruegg et al. J. Cell Biol. 177:179, 1991) and the extracellular matrix protein osteopontin (Bayless et al., J. Cell Sci. 111:1165-1174, 1988).

The expression of an α4 integrin may be measured using art-known methods including Western blot, ELISA, fluorescence-assisted cell sorting (FACS), proteomics, RT-PCR, Northern blot, and gene chip technology. The biological activity of α4 integrin may be measured in cellular based assays, including, but not limited to: JAK3 activation and phosphorylation, STAT6 activation and phosphorylation, and STAT6-responsive reporter plasmid assays (e.g., assays using a promoter which is activated by STAT6, e.g., IGE, MHC class II, and CD23). Additional assays for integrin antagonists are described below.

Examples of integrin antagonists are described in: U.S. Pat. Nos. 5,821,231; 5,869,448; 5,936,065; 6,197,794; 6,229,011; 6,265,572; 6,288,267; 6,329,372; 6,348,463; 6,362,204; 6,365,619; 6,380,387; 6,388,084; 6,423,728; 6,426,348; 6,445,550; 6,458,844; 6,479,666; 6,482,849; 6,596,752; 6,667,331; 6,668,527; 6,685,617; 6,806,365; 6,835,738; 6,855,706; 6,872,719; 6,878,718; 6,903,128; 6,911,451; 6,916,933; 7,015,216; 7,105,520; 7,153,963; 7,160,874; 7,193,108; 7,250,516; and 7,291,645 (each herein incorporated by reference); in U.S. Patent Application Publication Nos. 2002/0049236; 2003/0004196; 2003/0018016; 2003/0078249; 2003/0083267; 2003/0100585; 2004/0039040; 2004/0053907; 2004/0087574; 2004/0102496; 2004/0132809; 2004/0229858; 2004/0229859; 2006/0014966; 2006/0030553; 2006/0166866; 2006/0166961; 2006/0211630; 2006/0241132; 2007/0054909; and 2007/066533 (each herein incorporated by reference); in European Patent Nos. EP 0842943; EP 0842944; EP 0842945; EP 0903353; and EP 0918059; in PCT Publication Nos. WO 93/13798; WO 93/15764; WO 94/15958; WO 94/16094; WO 95/15973; WO 95/19790; WO 96/00581; WO 96/06108; WO 96/22966; WO 96/40781; WO 97/02289; WO 97/03094; WO 97/49731; WO 98/04913; WO 98/04247; WO 98/42656; WO 98/53814; WO 98/53817; WO 98/53818; WO 98/54207; WO 98/58902; WO 99/06390; WO 99/06431; WO 99/06432; WO 99/06433; WO 99/06434; WO 99/06435; WO 99/06436; WO 99/06437; WO 99/10312; WO 99/10313; WO 99/20272; WO 99/23063; WO 99/24398; WO 99/25685; WO 99/26615; WO 99/26921; WO 99/26922; WO 99/26923; WO 99/35163; WO 99/36393; WO 99/37605; WO 99/37618; WO 99/43642; WO 01/42215; WO 01/47868; WO 01/70670; and WO 02/28830. Examples of antibodies, blocking peptides, nucleic acid inhibitors, and small molecule inhibitors that can be used as integrin antagonists are described below.

### Antibodies

An integrin antagonist can be an antibody. Examples of antibodies include monoclonal antibodies, polyclonal antibodies, multispecific antibodies (e.g. bi-specific antibodies) formed from at least two intact antibodies, and antibody fragments so long as they function as an integrin antagonist. Other kinds of antibody included in the invention are antibody fragments (e.g., Fab, Fab′, Fv fragments, diabodies, linear antibodies, and single chain antibody molecules). Additional examples of antibodies include monoclonal antibodies that are chimeric, primatized, or humanized. The term antibody herein used also encompasses proteins or molecules which contain the amino acid sequence of the variable region of an immunoglobulin gene (e.g., an antibody mimic). Methods for the production of the various antibodies described above are known in the art.

Studies using a specific monoclonal antibody antagonist of α4 integrin have demonstrated that inhibitors of VLA-4 cell adhesion can inhibit or reduce the severity of numerous pathological conditions, including inflammatory, respiratory, and autoimmune conditions (Chisholm et al., Eur. J. Immunol. 23:682, 1993; Richards et al., Am. J. Resp. Cell Mol. Biol. 15:172, 1996; Fryer et al., J. Clin. Invest. 99:2036, 1997; Soiluhanninen et al., J. Neuroimmunol. 72:95, 1997). These pathological processes can also be inhibited with agents other than antibodies, as evidenced in animal model studies using synthetic CS-1 peptide or small molecule peptide inhibitors of VLA-4 (Ferguson et al., Proc. Natl. Acad. Sci., 88:8072, 1991; Wahl et al., J. Clin. Invest. 94:655, 1994; Molossi et al., J. Clin. Invest. 95:2601, 1995; Abraham et al., J. Resp. Crit. Care. Med. 156:696, 1997; and Jackson et al., J. Med. Chem. 40:3359, 1997).

An exemplary antibody that binds and blocks the biological activity of α4 integrin is natalizumab, a recombinant humanized IgG4k monoclonal antibody produced in murine myeloma cells (reviewed in Rudick and Sandrock, Expert Rev. Neurother. 4:571-580, 2004). Natalizumab contains human antibody framework regions and the complementarity-determining regions of a murine antibody that binds to α4-integrin.

Other compounds have been developed that can target and bind to α4 integrin in a manner similar to antibodies. Certain of these “antibody mimics” use non-immunoglobulin protein scaffolds as alternative protein frameworks for the variable regions of antibodies. For example, Ladner et al. (U.S. Pat. No. 5,260,203, herein incorporated by reference) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability, and are more easily modified.

The technique of Lipovsek et al. (U.S. Pat. Nos. 6,818,418 and 7,115,396, herein incorporated by reference) can also be used to create integrin antagonists. Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, a process for loop randomization and shuffling may be employed in vitro that is similar to the process of affinity maturation of antibodies in vivo.

Beste et al. (Proc. Natl. Acad. Sci. U.S.A. 96:1898-1903, 1999) also discloses techniques that can be used to create integrin antagonists. Beste et al. (supra) discloses an antibody mimic based on a lipocalin scaffold (i.e., an Anticalin®). Lipocalins are composed of a n-barrel with four hypervariable loops at the terminus of the protein. Beste et al. (supra) subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable for use as an alternative to antibodies. Anticalins® are small, single chain peptides, typically between 160 and 180 residues, which provide several advantages over antibodies, including decreased cost of production, increased stability in storage, and decreased immunological reaction. Thus, the structural framework of Anticalins® can be used to produce an integrin antagonist according to the present invention.

Hamilton et al. (U.S. Pat. No. 5,770,380, herein incorporated by reference) discloses a method of making synthetic antibody mimics that may be used as integrin antagonists. Hamilton et al. (supra) describes a method of making a synthetic antibody mimic using the rigid, non-peptide organic scaffold of calixarene, attached with multiple variable peptide loops used as binding sites. The peptide loops all project from the same side of the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore, it is less vulnerable to attack by protease enzymes. Neither does the scaffold consist purely of a peptide, DNA, or RNA, meaning this antibody mimic is relatively stable in extreme environmental conditions and has a long life span. Further, since the calixarene-based antibody mimic is relatively small, it is less likely to produce an immunogenic response.

Another class of antibody mimics that may be used to produce an integrin antagonists is disclosed in Murali et al., Cell. Mol. Biol. 49:209-216, 2003. Murali et al. (supra) discloses a methodology for reducing antibodies into smaller peptidomimetics, which may also be useful as an alternative to antibodies in the practice of the present invention.

In addition to non-immunoglobulin protein frameworks, antibody properties have also been mimicked in compounds that include RNA molecules and unnatural oligomers (e.g., protease inhibitors, benzodiazepines, purine derivatives, and beta-turn mimics), each of which may be used in the preparation of integrin antagonist suitable for use with the present invention.

### Blocking Peptides

Other exemplary integrin antagonists suitable for use with the present invention are blocking peptides that bind to and antagonize the activity of VLA-4 on leukocytes. The VLA-4 binding domain in the CS-1 region of fibronectin comprises the octapeptide: Glu-Ile-Leu-Asp-Val-Pro-Ser-Thr, as well as the overlapping pentapeptides Glu-Ile-Leu-Asp-Val and Leu-Asp-Val-Pro-Ser. Thus, the minimal amino acid sequence required for inhibition would be Leu-Asp-Val (LDV). In fact, the LDV minimal tripeptide sequence has been shown to be equally effective as the full length CS-1 fragment in binding the activated from of VLA-4 (Wayner et al., J. Cell Biol. 116:489, 1992). Another exemplary integrin antagonist suitable for use in the present invention are Arg-Gly-Asp (RGD) based cyclic peptides capable of inhibiting both α4β1 and α5β1 integrins from binding to fibronectin, as disclosed in Nowlin et al., J. Biol. Chem. 268:20352, 1993; and PCT/US91/04862. The tri- and tetra-peptide sequences IDA and REDV are suitable for use as blocking peptides in the present invention, as they have been shown to regulate fibronectin binding to VLA-4, while the pentapeptide QIDSP regulates binding of VLA-4 to VCAM-1 and is also suitable for use with the present invention (Humphries et al., Ciba Found. Symp. 189:177, 1995). Additional VLA-4 and VLA-5 blocking peptides that have been shown to inhibit a hypersensitivity response include GRDGSP and EILDV.

Ku et al. (Proc. Natl. Acad. Sci. U.S.A. 92:6552-6556, 1995) describes a method of creating peptide antagonists that may function as integrin antagonists. Ku et al. (supra) discloses a peptide antagonist based on cytochrome b562. In this method, Ku et al. generated a library in which two of the loops of cytochrome b562 were randomized and selected for binding against bovine serum albumin (BSA). The individual mutants were found to bind selectively with BSA similar to anti-BSA antibodies. Similar mutants can be constructed to selectively bind the α4 integrin subunit.

### Nucleic Acid Inhibitors

In some embodiments, the integrin antagonist is a nucleic acid inhibitor. For example, ribozymes, antisense RNA and/or interfering RNA (RNAi) molecules can be used to target α4 integrin subunit.

In some embodiments, RNAi molecules are used to target the α4 integrin subunit. In mammalian cells, the introduction of dsRNAs (e.g., dsRNAs having at least 10, 20, 30, 40, 50, or 50 or more nucleotides in length; and desirably having 21 nucleotides in length) often initiates a potent antiviral response, which is exemplified by nonspecific inhibition of protein synthesis and RNA degradation. The phenomenon of RNA interference is described and discussed, for example, in Bass, Nature 411:428-29, 2001; Elbahir et al., Nature 411:494-98, 2001; and Fire et al., Nature 391:806-11, 1998; wherein methods of making interfering RNA also are discussed. RNAi molecules targeting the α4 integrin may include all or part of a nucleic acid sequence the complement of which is substantially identical to the mRNA sequence of α4 integrin (SEQ ID NO: 12 or SEQ ID NO: 35). The nucleic acid sequence preferably has at least 10, 20, 30, 40, 50, or even 50 or more nucleotides in length (e.g., 21 or 25 nucleotides in length). The RNAi molecules can be made by methods known in the art. Exemplary RNAi molecules according to the invention could have up to 29 bps, 25 bps, 22 bps, 21 bps, 20 bps, 15 bps, 10 bps, 5 bps or any integer thereabout or therebetween. The RNAi molecule may have a sequence which is complementary to any sequence within SEQ ID NO: 12 or SEQ ID NO: 35 (e.g., a sequence that is complementary to nucleotides 1-25, 50-75, 50-100, 50-150, 50-1000, or 50-2000 of SEQ ID NO: 12 or SEQ ID NO: 35).

The RNAi can also comprise two complementary molecules, or can be constructed such that a single transcript has both the sense and complementary antisense sequences from the target sequence (e.g., SEQ ID NO: 12 or SEQ ID NO: 35) so that the RNAi molecule is capable of forming a hairpin.

Methods for designing double stranded RNA to inhibit gene expression in a target cell are known (e.g., U.S. Pat. No. 6,506,559; Elbashir et al., Methods 26:199-213, 2002; Chalk et al., Biochem. Biophysy Res. Comm 319:264-274, 2004; Cui et al., Comp. Method Prog. Biomed. 75:67-73, 2004; and Wang et al., Bioinformatics 20:1818-1820, 2004). For example, the design of RNAi molecules (including hairpins) typically follows known thermodynamic rules (see, e.g., Schwarz et al., Cell 115:199-208, 2003; Reynolds et al., Nature Biotechnol. 22:326-30, 2004; and Khvorova et al., Cell 115:209-16, 2003). Many computer programs are available for selecting regions of the α4 integrin sequence that are suitable target sites. These include programs available through commercial sources such as Ambion, Dharmacon, Promega, Invitrogen, Ziagen, and GenScript as well as noncommercial sources, such as EMBOSS, The Wistar Institute, Whitehead Institute, and others.

For example, design can be based on the following considerations. Typically shorter sequences, i.e., less than about 30 nucleotides are selected. The coding region of the mRNA is usually targeted. The search for an appropriate target sequence optionally begins 50-100 nucleotides downstream of the start codon, as untranslated region binding proteins and/or translation initiation complexes may interfere with the binding of the siRNP endonuclease complex. Some algorithms, e.g., based on the work of Elbashir et al., supra, search for a 23-nt sequence motif AA(N19)TT (N, any nucleotide) and select hits with approximately 50% G/C-content (30% to 70% G/C-content can also be used for selection). If no suitable sequences are found, the search is extended using the motif NA(N21). The sequence of the sense RNAi corresponds to (N19)TT or N21 (position 3 to 23 of the 23-nt motif), respectively. In the latter case, the 3′ end of the sense siRNA is converted to TT.

Other algorithms preferentially select RNAi molecules corresponding to the target motif NAR(N17)YNN, where R is purine (A, G) and Y is pyrimidine (C, U). The respective 21-nt sense and antisense RNAi therefore begin with a purine nucleotide and can also be expressed from polymerase III expression vectors without a change in targeting site; expression of RNAs from polymerase III promoters is only efficient when the first transcribed nucleotide is a purine.

Other nucleic acids, e.g., ribozymes or antisense, can also be designed based on known principles. For example, Sfold (see, for example, Ding et al., Nucleic Acids Res. 32 Web Server issue:W135-W141; Ding and Lawrence, Nucl. Acids Res. 31: 7280-7301, 2003; and Ding and Lawrence, Nucl. Acids Res. 20:1034-1046, 2001) provides programs relating to designing ribozymes and antisense, as well as RNAi molecules. Examples of antisense RNA molecules that target α4 integrin include a nucleic acid sequence that is complementary to a nucleic acid sequence that is substantially identical to all or part of the mRNA sequence of α4 integrin (SEQ ID NO: 12 or SEQ ID NO: 35), may consist of at least 10, 20, 30, 40, 50, 60, 70, 80, 90, 100, or even 100 or more nucleotides; and, in particular, may be complementary to nucleotides 1-25, 50-75, 50-100, 50-150, 50-1000, or 50-2000 of SEQ ID NO: 12 or SEQ ID NO: 35.

An additional class of nucleic acid inhibitors are peptide-nucleic acids. Peptide-nucleic acids are molecules which include a nucleic acid sequence that is complimentary to a sequence substantially identical to all or a part of the mRNA nucleic acid sequence of a target protein (e.g., an α4 integrin). Peptide-nucleic acids that are effective as integrin antagonists, have a nucleic acid sequence that is complimentary to a sequence substantially identical to all or a part of the mRNA of α4 integrin (SEQ ID NO: 12 or SEQ ID NO: 35), contain at least 10, 20, 30, 40, 50, 60, 70, 80, 90, 100, or even greater than 100 nucleotides; and promote a decrease in α4 integrin activity or expression. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 254, 1497 (1991).

### Small Molecule Inhibitors

Another class of integrin antagonists suitable for use with the present invention include small molecule inhibitors. Non-limiting exemplary small molecule inhibitors suitable for use with the present invention include: the CS-1 peptidomimetics disclosed in PCT Publication Nos: WO 96/15973 and WO 96/06108; U.S. Pat. Nos. 5,821,231; 5,936,065, and 5,869,448 (each herein incorporated by reference); the semi-peptidic inhibitors disclosed in PCT International Pub. No. WO 97/03094; the VLA-4 inhibitors disclosed in PCT International Pub. No. WO 96/22966, which uses the LDV tripeptide as a core group; cyclic peptides from 5 to 13 residues modeled after a portion of the CS1 peptide and containing a free acid and the closely related compounds disclosed in PCT International Publication Nos: WO 96/00581, WO 97/49731, and WO 96/20216; and the cyclic tetrapeptide dimers, which comprise cyclic dimeric peptides in which a peptide 1 and peptide 2 independently represent a tetrapeptide juxtaposed in parallel or antiparallel orientation by means of two linking moieties as disclosed in PCT International Pub. No. WO 97/02289.

Another class of integrin antagonists suitable for use with the present invention include the sulfonylated-Pro-Phe compounds as disclosed in U.S. Pat. No. 6,489,300 (herein incorporated by reference); sulfonylated dipeptide compounds disclosed in PCT International Publication No. WO 99/06437; dipeptide compounds disclosed in PCT International Publication Nos: WO 99/06432, WO 99/06433, and WO 99/06435; the tyrosine derivatives disclosed in PCT International Publication No. WO 98/54207; and the phenylalanine derivatives disclosed in U.S. Pat. No. 6,174,794 (herein incorporated by reference) and PCT International Publication Nos: WO 99/37618, WO 99/35163, and WO 99/43642; the substituted phenylalanine compounds disclosed in PCT International Publication No. WO 99/06431; the 4-amino-phenylalanine type compounds disclosed in PCT International Publication No. WO 99/06434; the N-aroylphenylalanine derivatives and closely related compounds disclosed in PCT International Publication Nos: WO 99/10312 and WO 99/10313; and the cyclic amino acids disclosed in PCT International Publication No. WO 99/26615 and the closely related compounds using β-amino acids disclosed in WO 98/153814, WO 99/26921, and WO 99/33789 are suitable for use in the present invention.

Another exemplary class of small molecule integrin antagonists include the 5-ring heterocycles and related compounds disclosed in European Patent Application Nos: EP 842 943, EP 842 944 and EP 842 945; and the heterocyclic amide compounds as disclosed in PCT International Publication No. WO 98/53814.

Additional small molecule inhibitors for use as integrin antagonist in the present invention are disclosed in, e.g., U.S. Patent Publication Nos: 2003/0130349 and 2002/0049236 (herein incorporated by reference), and PCT International Publication Nos: WO 98/04247, WO 98/04913, WO 99/37605, WO 99/36393, WO 99/24398, WO 98/42656, and WO 96/01544. In addition, the substituted anilides as disclosed in PCT International Publication No. WO 99/23063; the carbamoyloxy compounds disclosed in PCT International Publication No. 99/06390; the benzyl compounds disclosed in PCT International Publication No. WO 99/06436; the imidazolidine derivatives and substituted imidazoline derivatives disclosed in European Patent Application Nos: EP 903 353 and EP 918 059; the biarylalkanoic acids disclosed in WO 98/53817; the sulfonamide compounds as disclosed in WO 98/53818 and the closely related azapeptide acids as disclosed in WO 99/20272; the 4-substituted-4-piperidine carboxamide derivatives disclosed in PCT International Publication No. WO 99/25685; the substituted pyrrole derivatives disclosed in PCT International Publication No WO 99/26922; and the para-aminomethylaryl carboxamide derivatives disclosed in WO 99/26923 are also suitable for use with the present invention.

Another class of integrin antagonists suitable for use with the present invention include the conjugates comprising more than one integrin antagonist covalently attached to a polymer as described in U.S. Patent Publication No. 2006/0013799 (herein incorporated by reference).

Further small molecule inhibitors can be identified using screening or biological assays (e.g., ligand binding assays, protein or receptor activity assays, and other assays as known in the art and described herein). For example, an integrin antagonist may be identified by screening commercially available chemical or small molecule libraries.

Additional antibodies, blocking peptides, nucleic acid inhibitors, or small molecules shown to be potent inhibitors of α4 mediated integrin adhesion to VCAM-1, CS-1, or osteopontin, using the assays disclosed herein are suitable for use with the methods of the invention for the treatment of patients with multiple sclerosis as described herein.

### Functional Assays for Integrin Antagonists

A variety of assays are available to establish the antagonistic activity of an integrin antagonist for use in the compositions and methods of the present invention. Non-limiting exemplary assays include the Jurkat-endothelial cell adhesion assay and the Jurkat-CS-1 assay as disclosed in U.S. Patent Publication No. 2003/0130349 (herein incorporated by reference), and the EAE model disclosed in greater detail in Example 2. PCT International Publication No. WO 98/53817 further discloses an assay for determining antagonism of α4β7 dependent binding to VCAM-Ig fusion protein.

The Jurkat-endothelial cell adhesion assay measures the adhesive interactions of a T-cell line (Jurkat), which express the α4β1 integrin, to endothelial monolayers in the presence of test compounds to identify integrin antagonists suitable for use with the present invention. Briefly, the test compounds are added in increasing concentration to the T-cells, and then the T-cell/compound mixture is added to interleukin-1 stimulated endothelial cell monolayers. The plates are incubated, washed, and the number of attached T-cells is quantitated. The assay directly demonstrates the cell adhesion inhibitory or modulatory activity of integrin antagonists at various concentrations for use with the present invention.

The Jurkat-CS-1 assay, described in U.S. Patent Publication No. 2003/0130349 is a modification of the previously published method of Cardarelli et al. (J. Biol. Chem. 269:18668-18673, 1994; and Proc. Natl. Acad. Sci. U.S.A. 83:2647-2651, 1986). Briefly, a CS-1 peptide, CLHPGEILDVPST, and the scrambled control peptide CLHGPIELVSDPT are immobilized onto microplates using a heterobifunctional crosslinker (e.g., 3-(2-pyridyldithio)propionic acid N-hydroxysuccinimide ester (SPDP) as described by Pierschbacher et al., Proc. Nat. Acad. Sci. U.S.A. 80:1224-1227, 1983). Briefly, the procedure involves coating the microtiter plates with horse-serum albumin (HSA) for 2 hours, washing the plates, and derivitizing with 10 μg/ml SPDP for 1 hour. After washing the derivitized plates, a recently dissolved 100 μg/ml cysteine containing CS-1 or control peptide solution is added and allowed to crosslink to the plates overnight at 4° C. The unbound peptide is removed by washing and the unreacted sites are blocked with a 2.5 mg/ml solution of bovine-serum albumin (BSA). A known number of Jurkat cells in a defined volume (e.g., 100 μl of cells at 2.5×10⁶ cells/ml) are mixed with a desired concentration of the integrin antagonist and added to the peptide-coated dishes and incubated for 1 hour at 37° C. Following incubation, the plates are washed and attached cells are fixed with 3% paraformaldehyde in PBS and stained with toluidine blue overnight at room temperature. Cell attachment is quantitated via optical density at 590 nm using a vertical pathway spectrophotometer.

Preferred integrin antagonists are those which have low IC50 values in the Jurkat endothelial cell assay or the CS-1 assay, or at least moderate activity in both assays. Typically, an integrin antagonist suitable for use with the present invention has activity at less than 50 μM in the CS-1 assay or at less than 500 μM in the endothelial assay.

### Supplemental Therapeutic Agents for Use in the Combination Therapies of the Invention

In addition to an AFP (or a biologically active fragment, derivative, or analog thereof) and an integrin antagonist, the combination therapies of the invention can also include the administration of an antagonist (e.g., an antibody, blocking peptide, nucleic acid inhibitor, or small molecule) to CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6.

For example, the compositions of the invention may include, or the methods of the invention may involve the administration of, a CD80 antagonist as a supplemental therapeutic agent. CD80 provides a co-stimulatory signal to the T-cell receptor (Lanier et al., J. Immunol. 154:97-105, 1995). Myeloid dendritic cells from MS patients have increased CD80 expression (Karni et al., J. Immunol. 177:4196-4202, 2006); therefore, CD80 is an attractive target for treatment of MS.

The compositions of the invention may also include, and the methods of the invention may also involve the administration of, an antagonist of P-selectin. P-selectin is a protein which plays a role in recruitment of leukocytes to the site of injury and is also a target for MS therapy.

In addition, compositions of the invention may also include, and the methods of the invention may also involve the administration of, a sphingosine-1-phosphate-receptor-1 (S1P1) antagonist. S1P1 plays a role in thymocyte and lymphocyte maturation. An inhibitor of S1P1 has been shown to deprive thymocytes and lymphocytes of the signal to egress from lymphoid organs (Brinkman et al., Am. J. Transplant. 4:1019-1025, 2004). Therefore, an inhibitor of S1P1 can be included in the AFP/integrin antagonist treatment regimen described herein.

A hyaluronate receptor (CD44) antagonist can also be included in the compositions of the invention or administered with an AFP and integrin antagonist in the methods of the invention. Hyaluronate receptor is a protein which plays a role in leukocyte extravasation in inflammatory central nervous system disease (Brennan et al., Immunology 98:427-435, 1999). Hyaluronate receptor is also highly expressed in the T cells of MS patients (Soilu-Hanninen et al., J. Neuroimmunol. 166:189-192, 2005). Therefore, an antagonist of hyaluronate receptor can also be included in the combination therapies of the invention.

In another example, the compositions and methods of the invention may include a leukocyte function antigen-1 (LFA-1) antagonist. LFA-1 is expressed on lymphocytes and plays a major role in the activation and trafficking of T-lymphocytes to the site of inflammation. LFA-1 has also been considered as a therapeutic target for MS (Avolio et al., J. Neurol. Sci. 186:65-73, 2001; Lujan et al., Mult. Sclerosis 4:239-242, 1998); therefore, an antagonist of LFA-1 may also be included in the combination therapies of the invention.

An antagonist of CD11/CD18 may also be used in conjunction with the compositions and methods of the present invention. CD11/CD18 is a heterodimeric integrin receptor which consists of one of three α subunits (CD11a, CD11b, or CD11c) and one β subunit (CD18). The CD11/CD18 receptor is involved in crucial leukocyte adhesion functions, including chemotaxis, phagocytosis, adhesion to the endothelium, aggregation, and cell-mediated cytotoxicity. One humanized monoclonal antibody to CD11/CD18 is already in phase I MS clinical trials (Bowen et al., Clin. Pharmacol. Ther. 64:339-346, 1998).

Additionally, the compositions and methods of the invention may include CD20 antagonist. CD20 is a B-lymphocyte surface molecule which plays a role in the development and differentiation of B-cells into plasma cell. Monoclonal antibodies to CD20 include rituxumab, ibritumomab tiuxetan, and tositumomab. Rituximab (Rituxan®, by Genentech) is currently being studied in a multi-center phase II/III trial involving primary-progressive MS, and a phase II trial in secondary-progressive MS. A case report for the use of rituximab on MS was reported in Stuve et al. (Arch Neurol. 2005; 62:1620-1623). Therefore, an antagonist of CD20 can also be included in the combination therapies of the invention.

In another example, the compositions and methods of the invention may include a CD86 antagonist. CD86 (cluster of differentiation 86) is a protein that provides a costimulatory signal necessary for T cell activation and survival. CD86 principal mode of action is by binding to CD28. CD86 and CD80 provide the necessary stimuli to prime T cells against antigens presented by antigen-presenting cells. The CD86 pathway is currently being evaluated as a potential target for the treatment of MS. Therefore, an antagonist of CD86 can also be used in conjunction with the combination therapies of the invention.

The compositions and methods of the invention may also include an antagonist of ICOS ligand. ICOS ligand is a membrane-protein that is expressed on activated monocytes and dendritic cells. ICOS ligand functions as a costimulatory signal for T-cell proliferation and cytokine secretion and induces B-cell proliferation and differentiation into plasma cells. ICOS ligand may play an important role in mediating local tissue response to inflammatory conditions and may modulate the secondary immune response by co-stimulating memory T-cell function. Therefore, an antagonist to ICOS ligand can also be used in conjunction with the combination therapies of the invention.

The compositions and methods of the invention may also include an CCR2 (chemokine (C—C motif) receptor 2) antagonist. CCR2 is a chemokine receptor that mediates recruitment of both infiltrating macrophages and resident microglia to specific sites of central nervous system inflammation. ChemoCentryx has initiated a Phase 1 for the use of CCX915, a small molecule antagonist of CCR2, in the treatment of MS. Humanized anti-CCR2 antibodies are described in U.S. Pat. No. 6,406,865, herein incorporated by reference. Therefore, an antagonist to CCR2 can also be used in conjunction with the combination therapies of the invention.

Additionally, the compositions and methods of the invention may include a CXCR3 antagonist. CXCR3 is a chemokine receptor that is expressed on activated T lymphocytes and NK cells. CXCR3 regulates leukocyte trafficking and the binding of chemokines to CXCR3 induces various cellular responses, most notably integrin activation, cytoskeletal changes and chemotactic migration. CXCR3-ligand interaction attracts Th1 cells and promotes Th1 cell maturation. CXCR3 has been implacated for a role in the development of MS. Several antagonists of CXCR3 are known in the art, including, small molecules (e.g., those described in WO 06/088837) and humanized antibodies (e.g., those described in WO 05/030793). Therefore, an antagonist to CXCR3 can also be used in conjunction with the combination therapies of the invention.

Finally, the compositions and methods of the invention may also include a CCR5 antagonist. CCR5 is a chemokine receptor expressed on T cells, macrophages, dendritic cells and microglia. CCR5 may play a role in inflammatory responses to infection. A role for CCR5 in the pathogenesis of MS has been suggested (Trebst et al., Am. J. Pathol. 159:1701-1710, 2001). Several antagonists to CCR5 are known in the art, including small molecule inhibitors (see, e.g., EP 1 539 695) and humanized antibodies (see, e.g., U.S. Pat. No. 7,122,185, herein incorporated by reference). Therefore, an antagonist to CCR5 can also be used in conjunction with the combination therapies of the invention.

### Pharmaceutical Compositions

The present invention also relates to a pharmaceutical composition that contains a therapeutically effective amount of an AFP and/or an integrin antagonist. The active ingredients, AFP and an integrin antagonist, may be present in the same pharmaceutical composition (a single dosage form) or separate pharmaceutical compositions (separate dosage forms) to be administered coextensively or separately. In addition, the compsotion can include one or more different AFPs or integrin antagonists. The compositions can be formulated for use in a variety of drug delivery systems. One or more physiologically acceptable excipients or carriers can also be included in the compositions for proper formulation. Suitable formulations for use in the present invention are found in Remington's Pharmaceutical Sciences, Mack Publishing Company, Philadelphia, Pa., 17th ed., 1985. For a brief review of methods for drug delivery, see, Langer, Science 249: 1527-1533, 1990.

The pharmaceutical compositions can be formulated for parenteral, intranasal, topical, oral, or local administration, such as by a transdermal means, for prophylactic and/or therapeutic treatment. Commonly, the pharmaceutical compositions are administered parenterally (e.g., by intravenous, intramuscular, or subcutaneous injection), by oral ingestion, or by topical application at areas affected by MS. Thus, the invention features compositions for parenteral administration that include an AFP and/or an integrin antagonist dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents, and the like. The invention also features compositions for oral delivery, which may contain inert ingredients such as binders or fillers for the formulation of a tablet, a capsule, and the like. Furthermore, this invention features compositions for local administration, which may contain inert ingredients such as solvents or emulsifiers for the formulation of a cream, an ointment, and the like. In different embodiments of the invention, the AFP and the integrin antagonist may be administered in the same or separate compositions for administration via the same or two different routes of administration.

Compositions of the invention may be sterilized by conventional sterilization techniques or they may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of an AFP and/or an integrin antagonist, such as in a sealed package of tablets or capsules (e.g., a blister pack). The composition in solid form can also be packaged in a container for a flexible quantity, such as in a squeezable tube designed for a topically applicable cream or ointment.

The compositions of the invention containing an effective amount of an AFP and/or an integrin antagonist can be administered for prophylactic and/or therapeutic treatments. In prophylactic applications, compositions of the invention containing an AFP and/or an integrin antagonist are administered to a patient susceptible to or otherwise at risk of developing MS. Such an amount is defined to be a “prophylactically effective dose.” In this use, the precise amounts again depend on the patient's state of health, but generally range from about 0.5 mg to about 400 mg of an AFP per dose (e.g., 10 mg, 50 mg, 100 mg, 200 mg, 300 mg, or 400 mg per dose) and from about 0.1 mg to about 500 mg of an integrin antagonist per dose (e.g., 10 mg, 50 mg, 100 mg, 200 mg, 300 mg, 400 mg, or 500 mg per dose). A dose of the AFP and/or integrin antagonist can be administered prophylactically to a patient one or more times per hour, day, week, month, or year (e.g., 2, 4, 5, 6, 7, 8, 9, 10, 11, or 12 times per hour, day, week, month, or year). More commonly, a single dose per week of an AFP and/or an integrin antagoist is administered to a patient.

In therapeutic applications, compositions of the invention can be administered to a patient already suffering from MS in an amount sufficient to cure or at least partially arrest one or more of the symptoms of the disease and their complications. An amount adequate to accomplish this purpose is defined as a “therapeutically effective dose.” Amounts effective for this use may depend on the severity of the disease or condition and the general state of the patient, but may range from about 0.5 mg to about 400 mg of an AFP per dose (e.g., 10 mg, 50 mg, 100 mg, 200 mg, 300 mg, or 400 mg per dose) and from about 0.1 mg to about 500 mg of an integrin antagonist per dose (e.g., 10 mg, 50 mg, 100 mg, 200 mg, 300 mg, 400 mg, or 500 mg per dose).

In several embodiments, the patient may receive an AFP (with or without an integrin antagonist) in the range of about 0.5 to about 400 mg per dose one or more times per week (e.g., 2, 3, 4, 5, 6, or 7 or more times per week), preferably about 5 mg to about 300 mg per dose one or more times per week, and even more preferably about 5 mg to about 200 mg per dose one or more times per week. The patient may also receive a biweekly dose of an AFP in the range of about 50 mg to about 800 mg or a monthly dose of an AFP in the range of about 50 mg to about 1,200 mg.

In other embodiments, an AFP may be administered to a patient in a typical dosage range of about 0.5 mg to about 400 mg per dose per week, about 1.0 mg to about 300 mg per dose per week, about 5 mg to about 200 mg per dose per week, about 10 mg to about 100 mg per dose per week, about 20 mg to about 80 mg per dose per week, about 100 mg to about 300 mg per dose per week, or about 100 mg to about 200 mg per dose per week. An AFP may be administered in the range of about 0.5 mg to about 100 mg per dose every other day, preferably about 5 mg to about 75 mg per dose every other day, more preferably about 10 mg to about 50 mg per dose every other day, and even more preferably 20 mg to about 40 mg per dose every other day. An AFP may also be administered in the range of about 0.5 mg to about 100 mg per dose three times per week, preferably about 5 mg to about 75 mg per dose three times per week, more preferably about 10 mg to about 50 mg per dose three times per week, and even more preferably about 20 mg to about 40 mg per dose three times per week.

In several embodiments, the patient may receive an integrin antagonist (with or without an AFP) in the range of about 0.1 to about 500 mg per dose per one or more times per week (e.g., 2, 3, 4, 5, 6, or 7 or more times per week), preferably about 0.1 mg to about 400 mg per dose one or more times per week, or about 0.1 mg to about 300 mg per dose one or more times per week, more preferably about 1 mg to about 200 mg per dose one or more times per week, or most preferably about 5 mg to about 100 mg per dose one or more times per week. The patient may also receive a biweekly, triweekly, or monthly dose of an integrin antagonist in the range of about 0.1 mg to about 1.5 g, preferably a dose in the range of about 1 mg to about 1,000 mg, more preferably a dose in the range of about 5 mg to about 800 mg. Preferably, the dose of an integrin antagonist (e.g., natalizumab) is about 300 mg per dose every four weeks.

In some embodiments where the integrin antagonist administered is natalizumab, the patient receives a typical dosage in the range of about 15 μg to about 150 mg per dose per week, preferably about 1 mg to about 120 mg per dose per week, more preferably about 2 mg to about 100 mg per dose per week, and even more preferably about 5 mg to 80 mg per dose per week. In another embodiment, natalizumab is administered in the range of about 200 mg to 400 mg per dose every four weeks. The patient may also receive an AFP polypeptide in the range of about 0.5 mg to about 200 mg per dose per week, preferably about 5 mg to about 100 mg per dose per week, more preferably about 10 mg to about 80 mg per dose per week, and even more preferably about 20 mg to about 70 mg per dose per week.

A dose of the AFP and/or integrin antagonist can be administered therapeutically to a patient one or more times per hour, day, week, month, or year (e.g., 2, 4, 5, 6, 7, 8, 9, 10, 11, or 12 times per hour, day, week, month, or year). More commonly, a single dose per week of an AFP and/or an integrin antagonist is administered to a patient.

In non-limiting embodiments of the methods of the present invention, an AFP and an integrin antagonist are administered to a patient: continuously for 1, 2, 3, or 4 hours; 1, 2, 3, or 4 times a day; every other day or every third, fourth, fifth, or sixth day; 1, 2, 3, 4, 5, 6, 7, 8, 9, or 10 times a week; biweekly; 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 times a month; bimonthly; 1, 2, 3, 4, 5, 6, 7, 8, 9, or 10 times every six months; 1, 2, 3, 4, 5, 6, 7, 8, 9, 10, 11, 12, 13, 14, 15, 16, 17, 18, 19, or 20 times a year; or biannually. The AFP (or biologically active fragment thereof) and the integrin antagonist may be administered at different frequencies during a therapeutic regime (i.e., administered at a higher frequency in the later stages of MS (e.g., administered once a week in the initial stages of MS and administered three times a week a later stage of MS) or administered at a higher frequency in the early stages of MS (e.g., administered three times a week during the initial stages of MS and administered once a week at a later stage of MS)). In additional embodiments, the AFP and the integrin antagonist may be administered to a patient at the same frequency or at a different frequency.

The amount of integrin antagonist and AFP polypeptide required to achieve the desired therapeutic effect depends on a number of factors, such as the specific integrin antagonist(s) chosen, the mode of administration, and clinical condition of the recipient. A skilled artisan will be able to determine the appropriate dosages of integrin antagonist and AFP (or biologically active fragment thereof) to achieve the desired results.

The coadministration of an AFP and an integrin antagonist according to the methods of this invention refers to the use of the two active ingredients in the same general time period or using the same general administration method. It is not always necessary, however, to administer both at the exact same time. For instance, if an AFP and an integrin antagonist are administered to a patient suffering from MS in two separate pharmaceutical compositions, the two compositions need not be delivered to the patient during the same time period or even during two partially overlapping time periods. In some cases, the administration of the second agent (e.g., an AFP) may begin shortly after completion of the administration period for the first agent (e.g., an integrin antagonist, such as natalizumab), or vice versa. The time gap between the two administration periods may vary from one or more hours, days, weeks, or months. In some cases, one therapeutic agent (e.g., an AFP) may be administered first with the second (e.g., an integrin antagonist, such as natalizumab) in a separate time period, and subsequently administered without the second in a following period. A typical schedule of this type may require a higher dosage of the first therapeutic agent in the first, co-administration period, and a lower dosage in the second period, and vice versa. The same applies for the second agent.

Single or multiple administrations of the compositions of the present invention that include an effective amount of an AFP and/or an integrin antagonist can be carried out with the dose levels and the pattern being selected by the treating physician. The dose and administration schedule can be determined and adjusted based on the severity of MS in a patient, which may be monitored throughout the course of treatment according to the methods commonly practiced by clinicians or those described herein.

In addition to an AFP and/or an integrin antagonist, the composition may also include an antagonist to CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6. The antagonist to CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6 may be an antibody, a binding peptide, a nucleic acid inhibitor, or a small molecule inhibitor; which can be identified according to the same methods described above with regard to an integrin antagonist.

The patient may receive an antagonist to CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6 in the range of about 0.1 to about 500 mg per dose per one or more times per hour, day, week, or month (e.g., 2, 3, 4, 5, 6, or 7 times per hour, day, week, or month), about 0.1 to about 400 mg per dose one or more times per week, about 0.1 to about 300 mg per dose one or more times per week, about 1 to about 200 mg per dose one or more times per week, or about 5 to about 100 mg per dose one or more times per week. The patient may also receive a biweekly, triweekly, or monthly dose of an antagonist to CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6 in the range of about 0.1 mg to about 1.5 g, preferably a dose in the range of about 1 mg to about 1,000 mg, more preferably a dose in the range of about 5 mg to about 800 mg.

### Kits of the Invention

The invention also features kits for treating or reducing the symptoms of, or severity of, MS according to the combination treatment method of the present invention. The kits typically include a pharmaceutical composition containing an AFP and a pharmaceutical composition containing an integrin antagonist, each in a therapeutically effective amount for treating MS. In one example, effective amounts of an AFP and an integrin antagonist can be present in a single pharmaceutical composition. Optionally, the pharmaceutical composition(s) may contain one or more pharmaceutically acceptable excipients.

Preferably, the kits include multiple packages of the single-dose pharmaceutical composition(s) containing an effective amount of an AFP and/or an integrin antagonist. Optionally, instruments or devices necessary for administering the pharmaceutical composition(s) may be included in the kits. For instance, a kit of this invention may provide one or more prefilled syringes containing an effective amount of an AFP and one or more prefilled syringes containing an effective amount of an integrin antagonist. Alternatively, the kit may provide one or more prefilled syringes containing an effective amount of an AFP and tablets containing a dosage of an integrin antagonist. Furthermore, the kits may also include additional components such as instructions or administration schedules for a patient suffering from MS to use the pharmaceutical composition(s) containing an AFP and/or an integrin antagonist.

In addition to an AFP and/or an integrin antagonist, the kit may also include an antagonist of CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6. The antagonist of CD80, P-selectin, sphingosine-1-phosphate receptor-1, hyaluronate receptor, LFA-1, CD11/CD18, CD20, CD86, ICOS ligand, CCR2, CXCR3, CCR5, CD40, CD154, CD28, IL-23, IL-17, or IL-6 may be an antibody, a binding peptide, a nucleic acid inhibitor, or a small molecule inhibitor.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

The following examples are meant to illustrate the invention and should not be construed as limiting.

### EXAMPLES

The following two examples are provided by way of illustration only and not by way of limitation. Those skilled in the art will readily recognize a variety of non-critical parameters that could be changed or modified to yield essentially the same or similar results.

### Example 1

### Functional Test of a Recombinant AFP Using MOG-EAE Mouse Model

Efficacy experiments of a recombinant version of human AFP (recombinant human AFP, rhAFP, produced according to U.S. Patent Application Publication No. 20040098755) were performed in a mouse model in which experimental autoimmune encephalomyelitis (EAE) is induced by immunization of susceptible strains of mice with myelin antigen or peptides (myelin oligodendrocyte protein [MOG] or proteolipid protein [PLP]). This assay system is useful for determining the functionality of an AFP of this invention.

Purpose of Study: The purpose of these studies was to test compounds intended as therapeutics for MS, an autoimmune disease directly associated with the major histocompatibility complex (MHC) class II molecule HLA-DR2. The mouse experimental autoimmune encephalomyelitis (EAE) model was chosen for its relevance to human MS.

EAE Model Description and Features: Experimental Allergic Encephalomyelitis (EAE) is a demyelinating disease of the central nervous system. It serves as the animal model MS (Goverman, Lab. Anim. Sci. 46:482, 1996; and Paterson, Clin. Immunol. Rev. 1:581, 1981). EAE can assume an acute, chronic, or relapsing-remitting disease course that is dependent upon the method of induction and type of animal used. Disease induction results in escalating degrees of ascending animal paralysis. The resulting paralysis is debilitating, but not painful, and most animals will show some degree of recovery even from advanced stages of EAE. Paralysis usually begins with a weakened tail, gradually followed by hind limb weakness progressing to paralysis, and less frequently front limb paralysis. EAE disease progression can be monitored with a scoring system that starts with the normal condition and ends when the mice become moribund. Since the severity of the disease varies from animal to animal there is no way to reliably predict whether an animal will recover. As a result, close monitoring is needed in this animal model.

EAE can be induced with components of the central nervous system (Levine and Sowinski, J. Immunol. 110:139, 1973; and Fritz et al., J. Immunol. 130:1024, 1983) or peptides (Tuohy et al., J. Immunol. 140:1868, 1988; McFarlin et al., Science 179:478, 1973; and Linington et al., Eur. J. Immunol. 23:1364, 1993) and also via T cell transfer from one animal to another animal (Yamamura et al., J. Neurol. Sci. 76:269, 1986). Complete Freund's adjuvant (CFA) must be used with the proteins or peptides to effectively trigger the autoimmune response. CFA is often used in combination with pertussis toxin (Lee, Proc. Soc. Exp. Biol. Med. 89:263, 1955; and Kamradt et al., J. Immunol. 147:3296, 1991) to increase the efficiency of immunization. It is not possible to administer analgesics to lessen any pain that may be associated with the CFA injections, as most analgesics affect the immune response that is an essential component of the model (Billiau, J. Leukoc. Biol. 70:849, 2001; and Naiki et al., Int. J. Immunopharmacol. 13:235, 1991).

### Experimental Design and Methods

Induction of experimental MS-like disease syndrome: 50 female mice (C57BL6) between 6 and 8 weeks of age, were immunized subcutaneously on day 0 (left paralumbar region) and day 7 (right paralumbar region) with an emulsion (125 μg per mouse) of myelin oligodendrocyte glycoprotein (mMOG-35-55 peptide) in CFA containing heat-killed Mycobacterium tuberculosis H37RA. In addition, mice were given pertussis toxin (Ptx) intraperitonealy on days 0 and 2 post-immunization.

Disease monitoring: The initial signs of disease (weakened tail or paralysis) were observed beginning ˜10 days after the first immunization. Actively immunized mice were assessed daily through day 30 for clinical signs of EAE according to an established scale:

| | |
|---|---|
| 0 | No disease |
| 1 | Tail weakness |
| 2 | One or two weak hind limbs, sufficient to impair righting |
| 3 | One of two hind limb paralysis |
| 4 | One or two front limb paralysis |
| 5 | Moribund or dead |

The 50 mice were randomized into 5 groups of 10 mice each. One group of 10 animals received a saline injection to serve as an untreated EAE disease control. Four compounds were evaluated in the remaining 4 groups.

Mice were injected with 100 μl of test rhAFP or control material IP daily. These compounds are: 1-500 μg rhAFP or 1-500 mg human serum albumin (control). Furthermore, depleting antibodies to specific leukocyte subsets (e.g., CD4⁺ cells) are employed as additional control(s) in some studies.

Mice were used in this study to assess the effect of rhAFP on disease progression in an experimental model of MS (EAE). Without treatment it was expected that many of the animals would develop signs and symptoms of EAE, namely progressive encephalopathy and paralysis.

In addition to daily monitoring of the animals for disease progression over a 30-day time course, animals were sacrificed at the end of the study and central nervous system tissues (brain and spinal cord) were harvested for immunohistochemical analysis of infiltrating, disease-causing cells (i.e., CD4⁺ T cells).

Additionally, six to ten-day short-term studies were employed to assess the effect(s) of rhAFP administration on the induction phase of disease. In these shorter studies, draining lymph node cells were harvested for FACs analysis of immunologic cell subsets including but not limited to: T cells, CD4⁺ cells, regulatory T cells, and their activation markers. A fraction of harvested cells from each treatment group were assessed for in vitro proliferative response to a panel of stimuli to assess Ag-specific recall response to the immunizing antigen (Ag), MOG35-55, and Ag-nonspecific responses to a panel of mitogens (Concanavalin A, PHA, and LPS). Supernatants from cultures set-up in the same fashion are analyzed for cytokines (e.g., IL-2, IL-4, or IFNγ).

### Example 2

Effect of AFP and an Integrin Antagonist in MOG-EAE Mouse Model

The synergistic effect of recombinant human AFP and an integrin antagonist (e.g., an antibody, such as a surrogate anti-mouse antibody (e.g., an anti-VLA-4 antibody or a rat anti-mouse antibody, such as PS/2)) for treating EAE is tested in a study utilizing the MOG-EAE or PLP-EAE mouse model for MS.

The general experimental design is identical to Example 1. Briefly, 70 female mice (C57BL6) between 6 and 8 weeks of age are immunized subcutaneously on day 0 (left paralumbar region) and day 7 (right paralumbar region) with an emulsion (125 μg per mouse) of myelin oligodendrocyte glycoprotein (mMOG-35-55 peptide) in CFA containing heat-killed Mycobacterium tuberculosis H37RA.

The 70 mice are randomized into 7 groups of 10 mice each. One group of 10 animals receives a saline injection to serve as an untreated EAE disease control. Six different formulations are evaluated in the remaining 6 groups. The mice of group 1 receive a placebo; group 2 receives rhAFP at 10 μg/day; group 3 receives rhAFP at 100 μg/day; group 4 receives the integrin antagonist at 10 μg/day; group 5 receives the integrin antagonist at 100 μg/day; group 6 receives both rhAFP and the integrin antagonist at 10 μg/day and 10 μg/day, respectively; and group 7 receives both rhAFP and the integrin antagonist at 100 μg/day and 100 μg/day, respectively. The experimental design can alternatively include modifying (e.g., increasing or decreasing) the dosages of one or both of the rhAFP and the integrin antagonist during the administration period. For example, the mice could initially be administered a constant dose of rhAFP and an escalating dose (e.g., 0.1, 1.0, 10, 20, 50, or 100 μg/mouse given every other day) of the integrin antagonist (e.g., a mouse anti-α4 mAb) to determine if rhAFP enables the administration of a suboptimal dose of the integrin antagonist. As an alternative, the mice could initially be administered a constant dose of the integrin antagonist and an escalating dose of rhAFP.

The administration is by daily injections (interperitoneally or subcutaneously) from day 0 until the end of experiment at between days 40 and 60. All groups are scored daily for disease symptoms according to the scale as described in Example 1 for the duration of the study.

All mice are euthanized between days 40 and 60, and various organs and blood (e.g., spleen, knees, and hind and fore paws) are harvested for immuno-histochemistry and immunological analysis.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference in their entirety.

What is claimed is:

1. A method of treating a patient with multiple sclerosis comprising administering to said patient alpha-fetoprotein (AFP) or a biologically active fragment thereof and an integrin antagonist.

2. The method of claim 1, wherein said AFP or biologically active fragment thereof is recombinant human AFP.

3. The method of claim 1, wherein said AFP or biologically active fragment thereof is non-glycosylated.

4. The method of claim 1, wherein said integrin antagonist is an antibody, a blocking peptide, a nucleic acid inhibitor, or a small molecule inhibitor.

5. The method of claim 4, wherein said antibody is an anti-α4 integrin antibody.

6. The method of claim 5, wherein said anti-α4 integrin antibody is natalizumab.

7. The method of claim 1, wherein said method further comprises administering an antagonist of one or more of the following proteins: CD80, P-selectin, sphingosine-1-phosphate receptor-1, hyaluronate receptor, leukocyte function antigen-1 (LFA-1), CD11/CD18, CD20, CD86, ICOS ligand, CCR2, CXCR3, CCR5, CD40, CD154, CD28, IL-23, IL-17, and IL-6.

8. The method of claim 7, wherein said antagonist of CD80, P-selectin, sphingosine-1-phosphate receptor-1, hyaluronate receptor, leukocyte function antigen-1 (LFA-1), CD11/CD18, CD20, CD86, ICOS ligand, CCR2, CXCR3, CCR5, CD40, CD154, CD28, IL-23, IL-17, or IL-6 is an antibody, a blocking peptide, a nucleic acid inhibitor, or a small molecule inhibitor.

9. The method of claim 1, wherein said AFP or biologically active fragment thereof, or said integrin antagonist is administered intravenously, intramuscularly, orally, by inhalation, parenterally, intraperitoneally, intraarterially, transdermally, sublingually, nasally, through use of suppositories, transbuccally, liposomally, adiposally, intraocularly, subcutaneously, intrathecally, topically, or through local administration.

10. The method of claim 1, wherein said AFP or biologically active fragment thereof, or said integrin antagonist is administered one or more times hourly, daily, weekly, biweekly, or monthly.

11. The method of claim 1, wherein said AFP or biologically active fragment thereof, and said integrin antagonist are administered coextensively or separately.

12. The method of claim 1, wherein said AFP or biologically active fragment thereof, and said integrin antagonist are administered in separate dosage forms.

13. The method of claim 1, wherein said AFP or biologically active fragment thereof and said integrin antagonist are administered in the same dosage form.

14. The method of claim 1, wherein said AFP or biologically active fragment thereof and said integrin antagonist are administered via two different routes of administration.

15. The method of claim 1, wherein said AFP or biologically active fragment thereof and said integrin antagonist are administered via the same route of administration.

16. The method of claim 1, wherein said AFP or biologically active fragment thereof is administered in the range of 0.5 mg to 400 mg per dose.

17. The method of claim 1, wherein said integrin antagonist is administered in the range of 0.1 mg to 500 mg per dose.

18. The method of claim 1, wherein said AFP or biologically active fragment thereof is administered prior to said integrin antagonist.

19. The method of claim 1, wherein said AFP or biologically active fragment thereof is administered after said integrin antagonist.

20. The method of claim 1, wherein said administering results in a loss of or reduction in severity of one or more symptoms of multiple sclerosis.

21. The method of claim 20, wherein said one or more symptoms of multiple sclerosis are selected from the group consisting of tingling, numbness, tremors, loss of balance, weakness in one or more limbs, blurred or double vision, slurred speech, swallowing problems, paralysis, lack of coordination, cognitive difficulties, fatigue, muscle spasms, dizziness, breathing problems, and seizures.

22. A composition comprising an AFP or a biologically active fragment thereof and an integrin antagonist.

23. The composition of claim 22, wherein said composition further comprises an antagonist to one or more of the following proteins: CD80, P-selectin, sphingosine-1-phosphate receptor-1, hyaluronate receptor, leukocyte function antigen-1 (LFA-1), CD11/CD18, CD20, CD86, ICOS ligand, CCR2, CXCR3, CCR5, CD40, CD154, CD28, IL-23, IL-17, and IL-6.

24. The composition of claim 22, wherein said AFP or biologically active fragment thereof is recombinant human AFP.

25. The composition of claim 22, wherein said AFP or biologically active fragment thereof is non-glycosylated.

26. The composition of claim 22, wherein said integrin antagonist is an antibody, a blocking peptide, a nucleic acid inhibitor, or a small molecule inhibitor.

27. The composition of claim 22, wherein said integrin antagonist is natalizumab.

28. The composition of claim 22, wherein said composition is formulated for intravenous, intramuscular, oral, parenteral, intraperitoneal, intraarterial, transdermal, sublingual, nasal, transbuccal, liposomal, adiposal, intraocular, subcutaneous, intrathecal, topical, or through suppository, inhalation, or local administration.

29. The composition of claim 22, wherein said AFP or biologically active fragment thereof is in a dose of between 0.5 mg and 400 mg and said integrin antagonist is in a dose of between 0.1 mg to 500 mg.

30. A kit comprising an AFP or a biologically active fragment thereof and an integrin antagonist, and instructions for administration to said patient.

31. The kit of claim 30, wherein said AFP or biologically active fragment thereof is recombinant human AFP.

32. The kit of claim 30, wherein said APF or biologically active fragment thereof is non-glycosylated.

33. The kit of claim 30, wherein said integrin antagonist is natalizumab

34. The kit of claim 30, wherein said AFP or biologically active fragment thereof or said integrin antagonist is formulated for intravenous, intramuscular, oral, parenteral, intraperitoneal, intraarterial, transdermal, sublingual, nasal, transbuccal, liposomal, adiposal, intraocular, subcutaneous, intrathecal, topical, or through suppository, inhalation, or local administration.

35. The kit of claim 30, wherein said AFP or biologically active fragment thereof and said integrin antagonist are present in the same composition.

36. The kit of claim 30, wherein said AFP or biologically active fragment thereof and said integrin antagonist are formulated in separate compositions.

37. The kit of claim 30, wherein said AFP or biologically active fragment thereof and said integrin antagonist are formulated for two different routes of administration.

38. The kit of claim 30, wherein said AFP or biologically active fragment thereof and said integrin antagonist are formulated for the same route of administration.
